(19) United States
(12) Patent Application Publication
(10) Pub. No.: US 2012/0070870 A1
(43) Pub. Date: Mar. 22, 2012

(54) Methods and Molecules for Yield Improvement Involving Metabolic Engineering

(75) Inventors: Jeffrey C. Way, Cambridge, MA (US); Joseph H. Davis, Cambridge, MA (US)

(73) Assignee: Ginkgo BioWorks, Boston, MA (US)

(21) Appl. No.: 13/322,383

(22) PCT Filed: Jun. 1, 2010
(86) PCT No.: PCT/US10/36902

§ 371 Date: Nov. 23, 2011

Related U.S. Application Data
(60) Provisional application No. 61/182,839, filed on Jun. 1, 2009.

(51) Int. Cl. C12P 7/64; C12N 9/12; C12P 7/16; C12P 7/42; C12P 7/56; C12N 9/00; C12N 9/06

(52) U.S. Cl. 435/134; 435/183; 435/194; 435/191; 435/146; 435/139; 435/160

(57) ABSTRACT

The invention features methods and compositions relating to cells that have been engineered to reduce or eliminate proteins having enzymatic activity that interfere with the expression of a metabolic product.

### BACKGROUND OF THE INVENTION

In general, the invention relates to metabolic engineering of cells for the enhanced production of a cellular product.

Metabolic engineering involves the industrial production of chemicals from biological sources. Typically, a microbe such as a bacterium or a single-celled eukaryote is engineered to produce a compound in large amounts that is normally produced in small amounts or not at all. Examples of compounds produced by metabolic engineering include ethanol, butanol, lactic acid, various vitamins and amino acids, and artemisinin. Metabolic engineering generally involves genetic modification of a host organism, such as expression of foreign genes to make enzymes that synthesize compounds that may not be native to the host organism, overexpression of genes using strong promoters, introduction of mutations that alter allosteric regulation, and introduction of mutations that limit the production of alternative products.

It is generally desirable to produce compounds as cheaply and efficiently as possible. One major cost in metabolic engineering is the ‘feedstock’—the mixture of nutrients used in the medium in which the microbe grows. The feedstock typically includes a carbohydrate source, a source of fixed nitrogen, sources of sulfur, phosphorus, and so on, as well as any specific nutritional requirements. One significant problem in metabolic engineering is that even under conditions of product production, much of the feedstock is channeled into other metabolic pathways that contribute to growth of the organism and production of its biomass. A second problem is the cost of the feedstock itself, especially when the feedstock includes, in addition to a carbohydrate, molecules that fulfill auxotrophic requirements. Therefore, there is a need in the art to limit production of biomass during metabolic engineering and also to reduce the cost of the feedstock.

### SUMMARY OF THE INVENTION

The invention generally provides improved cells, molecules, and methods for synthesis of products by metabolic engineering. In a general embodiment, the invention provides an engineered cell that synthesizes a product more cost-effectively than current methods by making use of a cell with the following characteristics. The cell contains one or more proteins that include an enzymatic function with an engineered connection to a sequence that can promote degradation of the protein. The cell also includes a regulatory system such that upon addition or withdrawal of a regulatory factor, which may be a chemical, a protein, photons, temperature, or any other factor, the degradation of the protein is enhanced. As a result, the metabolism of the cell is altered so that the synthesis and/or secretion of a desired product is enhanced. In a further embodiment, the desired product is obtained from the cell or the medium. The enzymatic function may promote growth of the cell during an expansion phase or may allow the culturing and expansion of the cell with less or none of an expensive feedstock component.

In a preferred embodiment, the invention provides an engineered cell that contains a protein that includes an enzymatic function and a sequence that can promote degradation of the protein, a regulatory system such that upon addition or withdrawal of a regulatory factor, the degradation of the protein is enhanced, synthesis and/or secretion of a desired product is consequently enhanced, and wherein the enzyme is a catabolic enzyme.

In a preferred embodiment, the invention provides an engineered cell that contains a protein that includes an enzymatic function and a sequence that can promote degradation of the protein, a regulatory system such that upon addition or withdrawal of a regulatory factor, the degradation of the protein is enhanced, synthesis and/or secretion of a desired product is consequently enhanced, and wherein the enzyme is an anabolic enzyme.

In a preferred embodiment, the invention provides an engineered cell that contains a protein that includes an enzymatic function and a sequence that can promote degradation of the protein, a regulatory system such that upon addition or withdrawal of a regulatory factor, the degradation of the protein is enhanced, synthesis and/or secretion of a desired product is consequently enhanced, and wherein the enzyme is an anabolic enzyme.

In a preferred embodiment, the invention provides an engineered cell that contains a protein that includes an enzymatic function and a sequence that can promote degradation of the protein, a regulatory system such that upon addition or withdrawal of a regulatory factor, the degradation of the protein is enhanced, synthesis and/or secretion of a desired product is consequently enhanced, and wherein the cell is a bacterial cell.

In a preferred embodiment, the invention provides an engineered cell that contains a protein that includes an enzymatic function and a sequence that can promote degradation of the protein, a regulatory system such that upon addition or withdrawal of a regulatory factor, the degradation of the protein is enhanced, synthesis and/or secretion of a desired product is consequently enhanced, and wherein the cell is a fungal cell.

In a preferred embodiment, the invention provides an engineered cell that contains a protein that includes an enzymatic function and a sequence that can promote degradation of the protein, a regulatory system such that upon addition or withdrawal of a regulatory factor, the degradation of the protein is enhanced, synthesis and/or secretion of a desired product is consequently enhanced, and wherein the cell is an insect cell, a plant cell, a protozoan cell, or a mammalian cell.

In a preferred embodiment, the invention provides an engineered cell that contains a protein that includes an enzymatic function and a sequence that can promote degradation of the protein, a regulatory system such that upon addition or withdrawal of a regulatory factor, the degradation of the protein is enhanced, synthesis and/or secretion of a desired product is consequently enhanced, and wherein the regulatory system controls synthesis of the protein.

In a preferred embodiment, the invention provides an engineered cell that contains a protein that includes an enzymatic function and a sequence that can promote degradation of the protein, a regulatory system such that upon addition or withdrawal of a regulatory factor, the degradation of the protein is enhanced, synthesis and/or secretion of a desired product is consequently enhanced, and wherein the regulatory system controls synthesis of a second factor that controls the degradation of the protein.

In a preferred embodiment, the invention provides an engineered cell that contains a protein that includes an enzymatic function and a sequence that can promote degradation of the protein, a regulatory system such that upon addition or withdrawal of a regulatory factor, the degradation of the protein is enhanced, synthesis and/or secretion of a desired product is consequently enhanced, and wherein the sequence that can promote degradation of the protein includes an amino acid sequence that differs from the sequence Ala-Ala-Asn-Asp-Glu-Asn-Tyr-Ala-Leu-Ala-Ala (SEQ ID NO: 1) by at most four amino acid substitutions or deletions.

In a distinct class of embodiments, the invention provides an engineered cell that contains a protein that includes an enzymatic function and a sequence that can promote degradation of the protein, a regulatory system such that upon addition or withdrawal of a regulatory factor, the degradation of the protein is enhanced, wherein the enzymatic function in an amino acid biosynthetic function.

In a preferred embodiment, the invention provides an engineered cell that contains a protein that includes an enzymatic function and a sequence that can promote degradation of the protein, a regulatory system such that upon addition or withdrawal of a regulatory factor, the degradation of the protein is enhanced, wherein the enzymatic function is part of aromatic amino acid synthesis.

In a distinct set of embodiments, the invention provides an engineered cell that contains a protein that includes an enzymatic function and a sequence that can promote degradation of the protein, a regulatory system such that upon addition or withdrawal of a regulatory factor, the degradation of the protein is enhanced, wherein the enzymatic function is part of the tricarboxylic acid cycle.

In a distinct set of embodiments, the invention provides an engineered cell that contains a protein that includes an enzymatic function and a sequence that can promote degradation of the protein, wherein the enzymatic function is part of fatty acid synthesis, the oxidative pentose phosphate pathway, or glycolysis.

In a distinct set of embodiments, the invention provides an engineered cell that contains a protein that includes an enzymatic function and a sequence that can promote degradation of the protein, wherein the enzymatic function is a kinase, an acetyl-CoA-producing enzyme, an enzyme that joins two carbon-containing reactant molecules into a single, carbon-containing product molecule, and an allosterically regulated enzyme

In a distinct set of embodiments, the invention provides an engineered cell that contains a protein that includes an enzymatic function and a sequence that can promote degradation of the protein, wherein enzymatic function is pyruvate kinase, shikimate kinase, pyruvate dehydrogenase, citrate synthase, and DAHP synthase.

In a distinct set of embodiments, the invention provides an engineered cell that contains a protein that includes an enzymatic function and a sequence that can promote degradation of the protein, wherein the enzymatic function is hexokinase, glucokinase, glucose-6 phosphatase, glucose-6-phosphate dehydrogenase, glucose phosphate isomerase, phosphofructokinase, fructose bisphosphate aldolase, glyceraldehyde phosphate dehydrogenase, triose phosphate isomerase, phosphoglyceromutase, enolase, phosphoenolpyruvate carboxykinase, pyruvate kinase, pyruvate dehydrogenase, pyruvate decarboxylase, pyruvate-formate lyase, lactate dehydrogenase, pyruvate carboxylase, citrate synthase, aconitate hydratase, isocitrate dehydrogenase, 2-oxoglutarate dehydrogenase, dihydrolipoamide succinyltransferase, succinyl-CoA ligase, succinyl-CoA hydrolase, succinate dehydrogenase, fumarase, malate dehydrogenase, malate synthase, isocitrate lyase, 2-oxoglutarate synthase, glutamate synthase, glutamate dehydrogenase, acetate CoA-ligase, acetyl-CoA carboxylase, malonyl-CoA transferase, acyl-carrier protein acetyltransferase, glutamine synthase, pyrroline-5-carboxylase reductase, glutamate ammonia ligase, aspartate transaminase, ornithine carbamoyl-transferase, arginino-succinate synthetase, aspartate-carbamoyltransferase, arginino-succinate lyase, arginase, a tRNA charging enzyme, tyrosine transaminase, anthranilate synthase, prephenate dehydratase, prephenate dehydrogenase, chorismate mutase, chorismate synthase, 3-phosphoshikimate carboxyvinyltransferase, shikimate kinase, shikimate dehydrogenase, 3-dehydroquinate dehydratase, 3-dehydroquinate synthase, DAHP synthase, D-phosphoglycerate dehydrogenase, phosphoserine transaminase, phosphoserine phosphatase, glycerol kinase, PRPP synthase, histidinol dehydrogenase, glucosamine acetyltransferase, glycogen synthase, 6-phosphoglucose lactonase, phosphogluconate dehydrogenase, ribose-5-phosphate isomerase, carbamoyl phosphate synthase, isopentenyl-diphosphate isomerase, dimethylallyl transferase, mevalonate kinase, HMG-CoA reductase, NADP/NAD oxidoreductase, formate dehydrogenase, hydrogenase, nitrate reductase, nitrite reductase, farnesyl-trans-transferase, geranyl-trans-transferase, ATP phosphoribosyl transferase, amido-P-ribosyl transferase, and arginine decarboxylase.

In a related embodiment, the invention also features nucleic acids encoding proteins, in which the nucleic acid comprises a sequence encoding a protein having any of the above enzymatic activities.

In a distinct class of embodiments, the invention provides an engineered cell that contains a protein that includes an enzymatic function and a sequence that can promote degradation of the protein, a regulatory system such that upon addition or withdrawal of a regulatory factor, the degradation of the protein is enhanced, wherein the regulatory system involves expression of an anti-sense RNA.

In a distinct class of embodiments, the invention provides an engineered cell that contains a protein that includes an enzymatic function and a sequence that can promote degradation of the protein, a regulatory system such that upon addition or withdrawal of a regulatory factor, the degradation of the protein is enhanced, wherein the regulatory system controls the expression of a protein that promotes degradation of the artificial protein.

In a distinct class of embodiments, the invention provides an engineered cell that contains a protein that includes an enzymatic function and a sequence that can promote degradation of the protein, a regulatory system such that upon addition or withdrawal of a regulatory factor, the degradation of the protein is enhanced, wherein the regulatory system controls replication or segregation of a plasmid.

The invention also provides nucleic acids encoding proteins, wherein the nucleic acid comprises a sequence encoding an enzyme fused to a sequence that can promote degradation of the protein, wherein the enzyme is an amino acid biosynthetic protein, a protein in the tricarboxylic acid cycle, a glycolytic enzyme, a fatty acid biosynthetic enzyme, or an enzyme of the oxidative pentose phosphate pathway, and wherein the nucleic acid further comprises an engineered operable linkage to a regulatory element.

The invention also provides nucleic acids encoding proteins, wherein the nucleic acid comprises a sequence encoding a shikimate kinase enzymatic activity fused to a sequence that can promote degradation of the protein, and wherein the nucleic acid optionally comprises an engineered operable linkage to a regulatory element.

The invention also provides methods of production, in which a cell containing a protein that includes an enzymatic function with an engineered connection to a sequence that can promote degradation of the protein is induced to undergo a regulatory switch that promotes degradation of the protein, enhanced synthesis of a desired product results, and the product is obtained from the culture of the cell.

In a preferred embodiment, the invention also provides methods of production, in which a cell containing a protein that includes an enzymatic function with an engineered connection to a sequence that can promote degradation of the protein is induced to undergo a regulatory switch that promotes degradation of the protein, enhanced synthesis of a desired product results, the product is obtained from the culture of the cell, and the product is purified.

In a more preferred embodiment, the invention provides methods of production of shikimic acid, in which a cell containing a protein that includes an shikimate kinase enzymatic activity with an engineered connection to a sequence that can promote degradation of the protein is induced to undergo a regulatory switch that promotes degradation of the protein, enhanced synthesis of a desired product results, the product is obtained from the culture of the cell, and the product is purified.

By “amino acid biosynthetic function” is meant an enzymatic activity corresponding to a point in metabolism at or after a point of feedback inhibition by an amino acid.

By “essential gene” of a cell (e.g., microbe) is meant a gene that is required for growth of the cell for the production of a given product.

Other features and advantages of the invention will be apparent from the detailed description and from the claims.

### BRIEF DESCRIPTION OF THE DRAWINGS

FIGS. 1A and 1B are schematic drawings showing the use of regulated degradation to enhance production by metabolic engineering. FIG. 1A shows a genetic construction (1) that includes a transcriptional regulatory element (2), a translational element (3), a coding sequence for a protein of interest such as an enzyme (4), fused in-frame to a coding sequence for a peptide or protein element that promotes degradation (5), the fusion protein product (6) that includes an enzymatic element (large oval) and a degradation tag that can be recognized by a protein degradation system (small oval), a schematic metabolic pathway in which reactions are represented by arrows (7), with a particular reaction (8) catalyzed by the enzymatic element of the fusion protein, leading to production of an undesired product (diamond, 9), as well as an alternative pathway leading to production of a desired product (triangle, 10). FIG. 1B shows the behavior of the system in response to a regulatory change, in which the levels of the protein (6) are reduced or eliminated; the reaction leading to the undesired product is also reduced or eliminated, leading to enhanced production of the desired product.

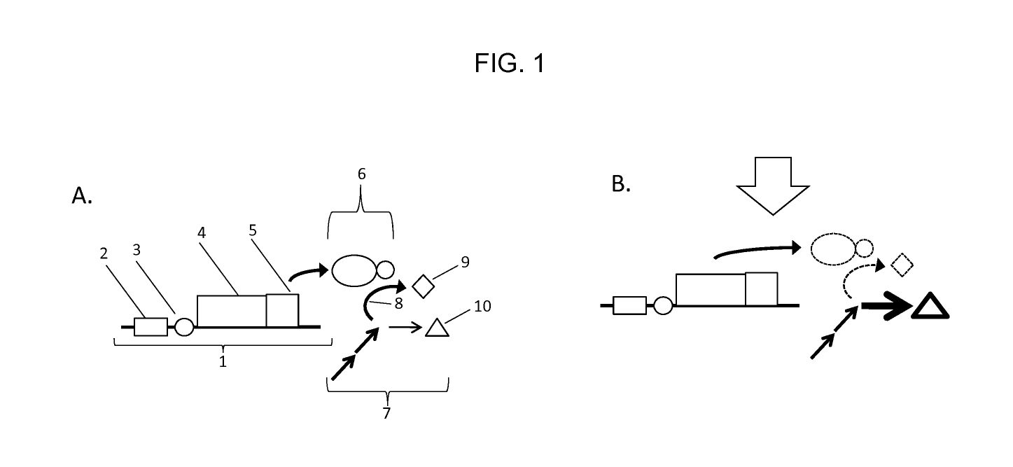

FIG. 2 is a schematic drawing showing an alternative metabolic pathway in which a desired product (triangle) is an intermediate in the production of an undesired product. In this configuration, the protein that is reduced upon a regulatory switch catalyzes a reaction that converts the desired product into another molecule. When the regulatory switch is activated, the protein is degraded and the desired product accumulates.

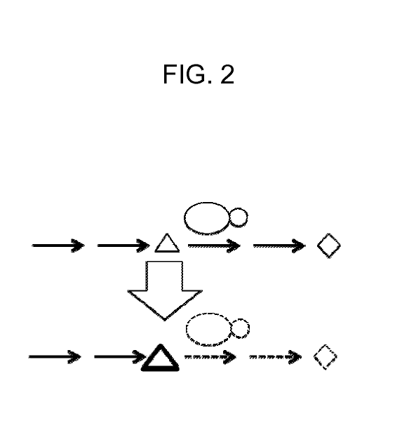

FIGS. 3A-3E are schematic drawings showing genetic constructions for regulating the degradation of a protein. FIG. 3A shows a DNA element (1) that includes a regulated promoter (2), a coding sequence for an enzyme of interest (3), and an in-frame coding sequence for a degradation tag (4). FIG. 3B shows a DNA element similar to that in FIG. 3A, except that it encodes an mRNA whose translation is regulated by a regulatory site within the mRNA (5). FIG. 3C shows a cellular configuration that includes a gene encoding a protein with a degradation tag, wherein the gene is transcribed, and also includes a second element in which the transcription of an antisense RNA is controlled by a regulated promoter (6). When the promoter is induced, the antisense RNA is expressed and binds to the mRNA encoding the protein with the degradation tag, blocking its translation and/or inducing its degradation, for example, by nucleases recognizing double-stranded RNA. FIG. 3D shows a cellular configuration that includes a gene encoding a protein with a degradation tag, wherein the gene is transcribed, and also includes a second element in which the transcription of a degradation factor is controlled by a regulated promoter (7). FIG. 3E shows a plasmid containing a gene encoding a protein with a degradation tag, and also containing an origin of replication that functions in a conditional manner (8).

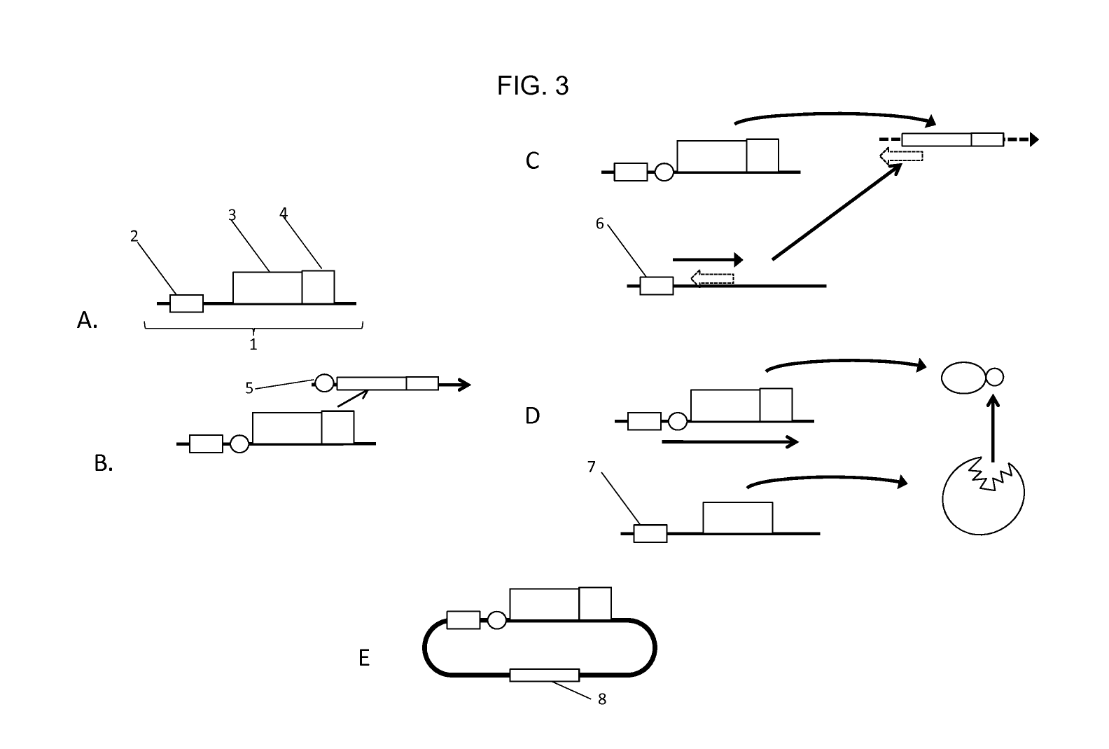

FIG. 4 is a schematic drawing showing a bacterial cell for production of L-Valine. The cell contains a plasmid encoding constitutive promoters (1, 5) driving transcription of ilvE (3) and panB (6) fused to ssrA degradation tags variants (4, 7). The protein product from each gene is translated using the encoded ribosome binding site (2). This plasmid contains a conditionally-replicated origin (8), allowing for facile curing of the plasmid (by a temperature shift, for example). The bacterial chromosome (9) contains mutations rendering the endogenous copies of ilvE (10) and panB (11) inactive.

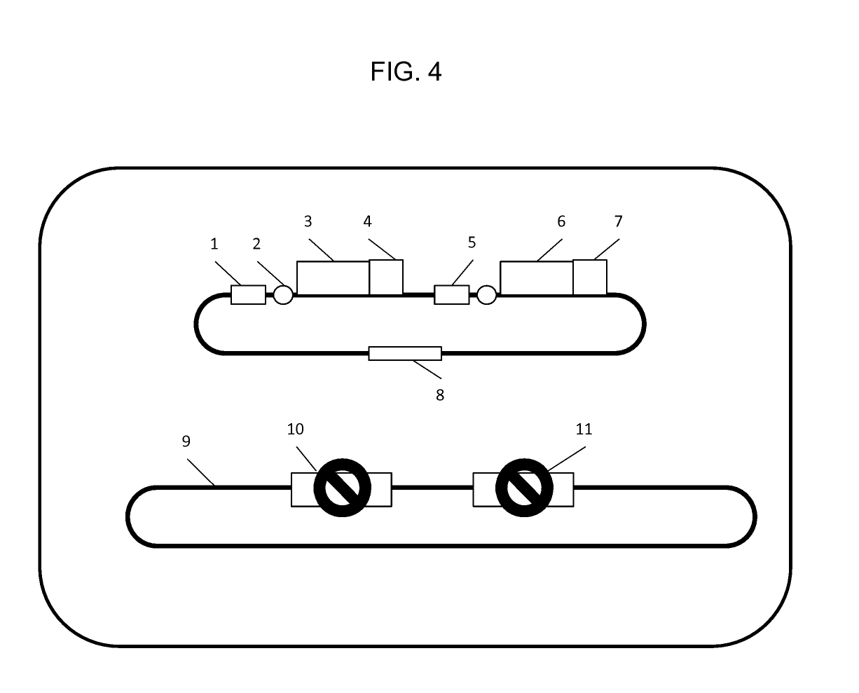

FIGS. 5A and 5B are schematic drawings showing a bacterial cell for production of L-Valine. Under permissive conditions (FIG. 5A), a conditionally-replicated plasmid (3) is maintained by a cell bearing loss-of-function chromosomal mutations (4) in specific metabolic enzymes. The plasmid encodes for the production of ssrA-tagged metabolic enzymes (1, 2) which complement the chromosomal mutants. Under the permissive conditions, production of these enzymes outpaces degradation resulting in a steady state pool of the protein products. Upon shifting to the restrictive conditions (FIG. 5B), the plasmid is lost from the cell, essentially terminating synthesis. Under these conditions, an energy-dependent protease (5) degrades the remaining ssrA-tagged protein products.

### DETAILED DESCRIPTION

A central aspect of the invention is the insight that it is useful and feasible to essentially harness the power of directed proteolysis to eliminate essential proteins during the production phase of metabolic engineering. To illustrate this insight, the generalized principles are described and exemplary schemes provided.

Broadly speaking, the methods of the invention control either the production, using regulated promoters, or degradation, using fused peptide segments which promote proteolysis (termed ‘degradation tags’), of one or more important or essential proteins. When a microbe carrying such a construction is to be grown to a large scale, conditions are created in which the rate of production of the protein of interest exceeds the combined rates of degradation and dilution (via cell growth and division) of said protein. Such ‘growth conditions’ produce sufficient steady-state concentrations of the protein of interest to allow for growth and replication of the microbe. When synthesis of a particular product is desired, the fermentation conditions are perturbed such that production is slowed and/or degradation is hastened resulting in depletion of the protein of interest. In general, the protein of interest is an enzyme that controls a major competing metabolic flux that does not contribute to the particular product. Depletion of such an enzyme results in increased flux through the desired metabolic pathway thereby enhancing the production efficiency of the product of interest.

In one instantiation of this technique, the protein of interest is fused to a degradation tag and its production is placed under the control of a regulated promoter. Under ‘growth conditions’, the promoter is induced such that production outpaces the basal levels of degradation. Upon switching to ‘production conditions’, the regulated promoter is repressed, thereby largely or completely terminating synthesis. Targeted protein degradation continues unabated until the protein of interest is essentially completely removed from the cell.

In an alternative configuration, the gene of interest may reside on a conditionally-replicated plasmid vector (bearing a temperature-sensitive origin, for example). Under the permissive conditions, the plasmid is maintained by the cell, allowing for robust synthesis of the protein of interest. Upon moving to non-permissive conditions, the plasmid is lost from the cell, essentially terminating synthesis of the protein of interest and, through the aforementioned degradation pathways, resulting in removal of this protein from the cell.

Those skilled in the art of genetic engineering will recognize that the specific features of this approach can be varied and yet produce the same general results. For example, many microbial protein degradation systems, or components thereof (e.g., adaptors, unfoldases, or proteases), are not essential, so an alternative configuration is to express a component of a protein degradation system from a regulated promoter and to express the protein of interest, fused to a degradation tag, from its native promoter or a weak, foreign promoter. In this configuration, the production of the protease component is repressed during the growth phase and induced during the production phase. Thus, protein degradation of the protein of interest is minimal during the ‘growth phase’ but can be induced during the ‘production phase.’ This configuration has the advantage of allowing for the use of a native promoter to drive production of the targeted essential protein. Such an approach need not be limited to the endogenous degradation machinery. Foreign degradation components derived from other organisms may be introduced into the strain of interest and utilized as described above. Such approaches obviate the need to perturb the endogenous degradation system, extending the generality of the system to microbes such as S. cerevisiae in which such a degradation system (i.e., the 26S proteasome) is essential. Indeed, Grilly et. al. have demonstrated the efficacy of E. coli-derived degradation machinery expressed in Saccharomyces cerevisiae and generated a strain that allows for targeted, controlled degradation of suitably tagged proteins in S. cerevisiae (Grilly et al. Mol Syst Biol 3:127 [2007]). Additionally, degradation tags have been identified for multiple energy-dependent proteases including ClpAP, ClpXP, HslUV, and Lon (Gur et al. PNAS 106:44 18503-18508 [2008], Gur et al. PNAS 105:42 16113-16118 [2008], Burton et al. Nat Struct Mol Bio 12(3):245-251 [2005], Flynn et al. Mol Cell 11(3):671-683). As such, addition of the appropriate tag to the protein of interest allows for targeted degradation via each of these proteases in a variety of organisms.

When a cell is configured to express an inducible degradation factor with a protein of interest fused to a degradation tag and expressed from a distinctly regulated promoter, under some circumstances the degradation of the protein of interest is inadequate due to continued expression. In such circumstances, it is often useful to express an anti-sense RNA that can inhibit translation of the protein of interest, for example from the same inducible promoter that regulates the degradation factor.

Finally, the production of proteolysis inhibitors or activators may be regulated, either using inducible promoters or conditionally-replicated plasmids, such that targeted degradation is inhibited during the ‘growth phase’ and permitted during the ‘production phase’. These alternative configurations illustrate that the general strategy of causing the disappearance of a protein during a ‘production phase’ may be implemented in various ways.

To allow for facile induction and repression of the genetic components (e.g., the degradation tagged gene of interest or a component of the degradation system), growth-phase-dependent promoters may be utilized. The E. coli promoter, osmY, is known to be strongly induced during stationary phase. The use of this, or a similarly regulated promoter, to drive production of a degradation component would allow for minimal degradation during culture growth (exponential phase) and efficient degradation once the culture had been saturated (stationary phase). As such, the gene of interest could be present during growth of the culture and later depleted allowing for efficient production of the small molecule of interest.

Alternatively, an exponential-phase promoter may be used to drive production of the protein of interest. During growth, production would outpace degradation, allowing for sufficient steady-state levels of this protein to support growth. Upon entering stationary phase, this promoter would be down-regulated, slowing production and allowing for degradation to remove the protein from the cell, thereby terminating growth and improving the production efficiency of the molecule of interest. The principles of the invention may also be applied in a eukaryotic system.

For example, yeasts are often used in the production of ethanol from a carbohydrate. In general, ethanol formation is promoted by pyruvate decarboxylase, while use of carbon for biomass production is promoted by the pyruvate dehydrogenase complex. Accordingly, to enhance the efficiency of ethanol production in yeast, pyruvate dehydrogenase is manipulated as follows. A chromosome gene encoding a subunit of the pyruvate dehydrogenase complex (PDH) is knocked out according to standard procedures. The corresponding gene is placed under control of a regulated promoter, such as a GAL1 promoter, GAL7 promoter or GAL10 promoter, which are inducible by galactose, or the CUP1 promoter, which is inducible by copper, zinc and other metal ions. The coding sequence for the subunit of the pyruvate dehydrogenase complex is also fused to a sequence encoding a protein segment that promotes ubiquitination. For example, an F box protein segment is used as a fusion partner to promote degradation of the subunit of the PDH. Zhou et al. (Molecular Cell [2000] 6:751-756, the entirety of which is incorporated by reference) describe how to construct an F box fusion to a second protein and express the protein in yeast and also in mammalian cells. In a specific illustration, a CUP1(promoter)-Fbox-PDH subunit genetic construction is placed in a yeast cell with a knockout of the corresponding chromosomal gene encoding the PDH subunit, the yeast cell is grown in the presence of an inducing metal ion, the inducing metal ion is withdrawn, and enhanced ethanol production results.

### Production of Lactic Acid

In scaled-up conditions for production of chemicals, it is typical to use low-cost carbohydrate sources such as glucose, sucrose, molasses, high-fructose corn syrup, depolymerized cellulosic biomass, or glycerol as a carbon source. To produce cellular constituents such as amino acids and fatty acids, much of the carbon flux from such carbon sources goes through pyruvate and acetyl-CoA. The latter molecule is the starting point for both the citric acid cycle (also known as the TCA cycle or the Krebs cycle), as well as fatty acid synthesis. Thus, when glucose or an equivalent molecule is used as a carbon source, the process for converting pyruvate to acetyl-CoA is an essential process for growth of typical organisms used in metabolic engineering such as yeast or E. coli.

According to the invention, for example, when the goal is to produce a lactic acid, it is useful to eliminate the competing reaction of the conversion of pyruvate to acetyl-CoA. It is generally not useful to simply mutate the gene or genes involved in this process, as they are often important or essential during the organism's growth phase. In the specific case of E. coli, two major systems exist for converting pyruvate to acetyl-CoA: pyruvate dehydrogenase and pyruvate-formate lyase. Mutational inactivation of both of these systems prevents growth on glucose as a sole carbon source. According to the invention, one of these systems, such as pyruvate-formate lyase (which functions under anaerobic conditions) is mutated, and pyruvate dehydrogenase is engineered to be active under conditions of growth, but is then post-translationally inactivated. Two specific methods of inactivation are provided by the invention, degradation by proteolysis and enzyme-mediated chemical modification such as phosphorylation. These forms of post-translational modification are optionally inducible and are preferably induced when switching from growth conditions to production conditions. It is also generally useful to turn off transcription of the relevant genes upon switching to production conditions.

In a specific embodiment of the invention, the proteolysis method may be employed as follows. Many bacteria, including E. coli, possess compartmentalized, energy-dependent proteases that recognize their substrates via short, fused peptide tags. Experiments in vitro and in vivo have shown that incorporation of such tags into foreign proteins is sufficient to direct efficient proteolysis of the targeted protein. The best characterized tag, ssrA, is derived from a system for degrading incorrectly translated proteins. Said system involves the ssrA tag sequence (Ala-Ala-Asn-Asp-Glu-Asn-Tyr-Ala-Leu-Ala-Ala in E. coli; SEQ ID NO: 1), an adaptor protein encoded by sspB that recognizes the ssrA-encoded peptide, and a series of downstream-functioning proteins (ClpX, ClpA, and ClpP) that unfold and degrade the tagged protein (Sauer et al., Cell 119:9-18 [2004]; Flynn et al., PNAS 98:10584-10589 [2001]). Normally, this ssrA tag sequence is incorporated into partially translated proteins where the ribosome has stalled due to a truncated or otherwise defective mRNA. According to the invention, this sequence or a variation thereof is incorporated into a protein of interest such as pyruvate dehydrogenase at the C-terminus. In one variation of the invention, the DNA sequence encoding the pyruvate dehydrogenase-ssrA fusion protein is expressed from an inducible/repressible promoter, and is repressed upon switching engineered bacteria from growth conditions to production conditions. Without wishing to be bound by theory, the pyruvate dehydrogenase-ssrA fusion protein is degraded at a constant rate, and when the transcription of the gene is halted, the mRNA naturally decays and the protein also decays due to the ssrA tag. According to the invention, the user may choose from a wild-type tag or various mutant tags, depending on the desired efficacy of binding between the protease and the substrate. Since the degradation rate of a protein-ssrA fusion will vary somewhat as a function of the protein sequence and the intracellular substrate concentration, some routine experimentation is required to identify an optimal ssrA degradation tag.

Interestingly, experiments have demonstrated that the adaptor protein, SspB is strictly required for efficient degradation of proteins bearing some mutant ssrA tags (for example, AANDENYADAS; SEQ ID NO: 2) (McGinness et al., Mol. Cell 22(5):701-707 [2006]). According to the invention, an alternative configuration is the regulated expression of SspB in a strain in which the chromosomal copy of pyruvate dehydrogenase has been fused to the mutated ssrA tag. In this way, the native control elements of pyruvate dehydrogenase remain unperturbed.

Extending this idea, adaptors from other bacteria (C. crescentus CC_{—}2101, for example) have been identified which bind their cognate ssrA tags (AANDNFAEEFAVAA in C. crescentus; SEQ ID NO: 3) and are capable of delivering bound substrates to E. coli ClpXP for degradation (Chien et al., Structure 15(10):1296-1305; Griffith et al., Mol Microbiol 70(4):1012-1025; Chowdhury et al., Protein Science 19(2):242-254). Critically, variants of these foreign tags are not bound by the E. coli SspB variant allowing for control of suitably tagged substrates via the foreign adaptor. According to the invention, the chromosomal copy of pyruvate dehydrogenase is fused to such a degradation tag. The cognate adaptor is then introduced on a plasmid vector under the control of a regulated promoter. Pyruvate dehydrogenase is targeted for degradation only under conditions in which the foreign adaptor is produced. In this manner, both the endogenous protease system and control elements of pyruvate dehydrogenase remain unperturbed.

The aforementioned methods require fusion of the degradation tag to the C-terminus of the protein of interest. Experiments have shown that proteins can also be targeted for degradation by ClpXP via N-terminal degradation tags (Flynn et. al., Mol Cell 11(3):671-683). Thus, according to the invention, one may alternatively fuse N-terminal degradation tags to the protein of interest (for a representative example, see λO tag, below). Additionally, ClpAP is known to degrade proteins bearing an N-end rule residue (i.e., Leu, Tyr, Trp, or Phe) at their N-terminus. Fusion of endoprotease recognition sites which, when cleaved give rise to one of these N-end rule residues, may also be used to target proteins for degradation via the N-terminus (Wang et al., Genes Dev 21(4):403-408). For simplicity, the following discussion will focus on a single implementation in which the protein of interest is targeted for degradation via fusion to an unmodified E. coli ssrA tag. Any other tag or degradation system may also be utilized.

Sample degradation tags include those listed in Table 1.

TABLE 1

| | |
|---|---|
| Wild-type E. coli ssrA tag: | AANDENYALAA |
| | (SEQ ID NO: 1) |
| Mutant 1: | AANDENYADAA |
| | (SEQ ID NO: 4) |
| Mutant 2: | AANDENYAAAA |
| | (SEQ ID NO: 5) |
| Mutant 3: | AANDENYAVAA |
| | (SEQ ID NO: 6) |
| Mutant 4: | AANDENYALDA |
| | (SEQ ID NO: 7) |
| Mutant 5: | AANDENYALVA |
| | (SEQ ID NO: 8) |
| Mutant 6: | AANDENYALAG |
| | (SEQ ID NO: 9) |
| Mutant 7: | AANDENYALGG |
| | (SEQ ID NO: 10) |
| Adaptor-dependent tag | AANDENYADAS |
| | (SEQ ID NO: 2) |
| Wild-type C. crescentus | AANDNFAEEFAVAA |
| ssrA tag: | (SEQ ID NO: 3) |
| ccSsra Specificity | ADNDNFAEEFADAS |
| Mutant 1: | (SEQ ID NO: 11) |
| λO tag (N-terminal tag) | MTNTAKILNFGRAS |
| | (SEQ ID NO: 12) |

At low substrate concentrations, the mutant tags allow for a reduced rate of intracellular degradation relative to the wild-type tag.

For the case of lactic acid production, the result is that after switching to a medium that represses synthesis of the pyruvate dehydrogenase-ssrA protein, this protein is degraded over a period of 2-60 minutes depending on the needs of the user, and metabolic flux of carbon into acetyl-CoA from pyruvate essentially ceases. As a result, flux through lactate dehydrogenase is increased. The method of the invention may be employed in combination with other engineering steps that enhance production of lactic acid, such as overproduction of lactate dehydrogenase, mutation of the zwf gene, growth in anaerobic conditions, and so on.

Metabolic engineering techniques to improve the biological production of amino acids have been applied with great success to the microbes B. subtilis, C. glutamicum, and E. coli. Using directed approaches, genes encoding enzymes that catalyze off-pathway reactions have been removed from the production strain allowing for increased metabolic flux through the pathway of interest. Additionally, random mutagenesis and selected breeding approaches have resulted in strains that overproduce the amino acid of interest (Park et al. PNAS [2007] 104(19):7797-7802). Mapping of said mutant strains often reveals that genes catalyzing off-target reactions have been inactivated confirming the efficacy of this approach. Oftentimes, the off-target pathways catalyze the production of alternative amino acids and thus inactivation of these genes results in strains auxotrophic for a variety of amino acids.

According to the invention, it is both useful and feasible to control the degradation of essential enzymes which catalyze these off-target reactions. Such controlled degradation approaches allow for growth of the strain under conditions in which these targeted enzymes are present and active, relieving the requirement for amino acid supplemented media. Upon changing to conditions of robust degradation or limited production, the targeted enzyme is depleted from the cell, resulting in increased metabolic flux through the pathway of interest and efficient production of the amino acid of interest.

In E. coli and the industrially relevant microbe C. glutamicum, production of the branched amino acids, L-Leucine, L-Valine and the coenzyme A precursor, pantothenate all utilize the metabolic intermediate, 2-ketoisovalerate. This intermediate is channeled to L-Leucine through the enzyme leuA, to L-Valine through ilvE and to panthonate through panB. According to the invention, when overproduction of L-Leucine is desired, ilvE and panB are targeted for degradation as follows. A plasmid bearing a temperature-sensitive origin as well as ssrA-tagged variants of ilvE and panB driven by a constitutive promoter is transformed into a host strain in which ilvE and panB have been knocked out of the chromosome. Under growth conditions, the plasmid is maintained and production outpaces degradation. Upon conversion to production conditions, the plasmid is cured from the cell, thereby effectively terminating synthesis and allowing for degradation to remove these enzymes from the cell. As such, metabolic flux is diverted toward the production of L-Leucine. Alternatively, when L-Valine production is desired, leuA and panB are targeted for degradation as described above. Critically, such approaches obviate the need to supplement the growth media with expensive amino acids (for example, ilvE-strains are auxotrophic for L-Valine and L-Isoleucine) while maintaining the ability to overproduce the small molecule of interest. A variety of other loss-of-function mutations are known to increase production of said amino acids (reviewed in Park, Lee Appl. Micribiol. Biotechnol. [2010] 85:491-596). According to the invention, such genes are targeted for degradation using the aforementioned approaches, allowing for efficient production of the desired amino acid under degradative conditions and robust cell growth on non-supplemented media under non-degradative conditions.

### Shikimic Acid Production

Another example further illustrates the invention. Shikimic acid is an intermediate in aromatic amino acid synthesis, and is also used in the chemical synthesis of the drug Tamiflu® as well as in combinatorial chemical libraries. The pathway for aromatic amino acid synthesis is illustrated below.

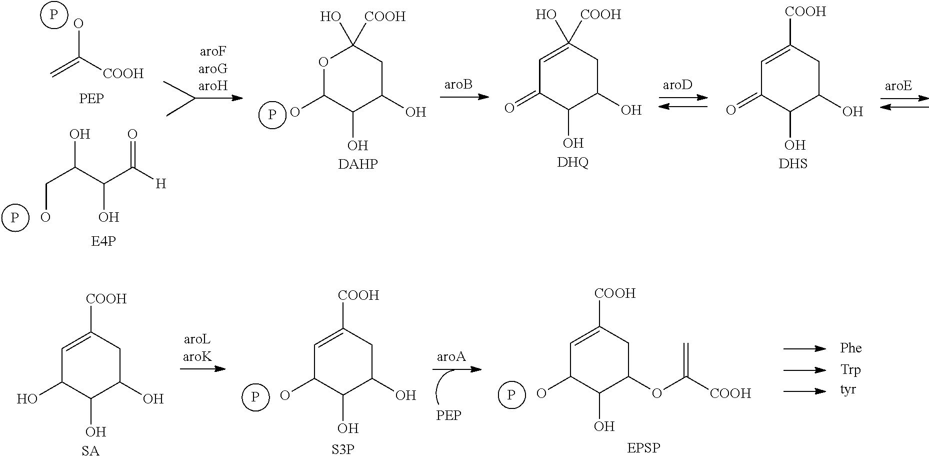

In brief, phosphoenolpyruvate and erythrose-4-phosphate, both from central metabolism, are condensed to a single 7-carbon intermediate that is processed through a series of intermediates that ultimately diverge into separate pathways for phenylalanine, tryptophan, and tyrosine. Shikimic acid is produced by the aroE gene product, and is then converted to shikimate phosphate by shikimate kinase, which in E. coli is produced independently by two genes, aroL and aroK. Current methods for producing shikimic acid involve the null mutation of both aroL and aroK, blocking shikimate phosphate production and leading to accumulation of shikimic acid. The aroK aroL double mutant is auxotrophic for tryptophan, tyrosine, and phenylalanine, each of which is an expensive molecule that must be added to the feedstock when shikimic acid is produced by metabolic engineering.

According to the invention, a shikimic acid-producing strain may be engineered as follows. One of the shikimate kinase genes, e.g., aroL, is knocked out by standard procedures. The other, e.g., aroK, is expressed with an ssrA peptide fused to its C-terminus. This fusion protein is expressed from a regulated promoter, such as the lac promoter, a quorum-sensing promoter, a promoter that is repressed in low-fixed nitrogen, a promoter that is induced by growth on glucose and repressed by growth on glycerol, or any other promoter that works well in the chosen conditions for switching from a growth mode to a production mode. In this way, the use of tyrosine, tryptophan, and phenylalanine can be avoided.

This control of shikimate kinase levels can be coupled to other strategies to enhance shikimic acid production, some of which are known in the art of metabolic engineering. For example, in E. coli, transport of glucose or most other carbohydrates normally involves transfer of a phosphate from phosphoenolpyruvate onto glucose. It is often useful to employ an alternative system using a protein that mediates facilitated diffusion of glucose and related carbohydrates, instead of the PEP-dependent system; a gene such as the glf gene from Zymomonas mobilis is often used. One common method is to knock out the endogenous ptsI gene and instead express the glf gene. According to the invention, an alternative method is to express a ptsI-ssrA fusion protein from a regulated promoter, and to also constitutively express the glf gene.

It is also useful to mutate genes encoding proteins that produce alternative products such as quinic acid. Further, it is useful to inactivate the shikimate transporter gene shiA by mutation, thus preventing re-uptake of shikimate that has been secreted. These approaches are based on Kraemer et al. (Metabolic Engineering 5:277-283 [2003], incorporated by reference herein), which reviews these established techniques and strategies.

According to the invention, in addition to blocking function of shikimate kinase, it is often useful to block conversion of PEP to pyruvate, which is normally catalyzed by the enzyme pyruvate kinase. Accordingly, a pyruvate kinase-ssrA fusion protein is expressed from a regulated promoter and the wild-type pyruvate kinase gene is inactivated. The result is accumulation of PEP, which is then used by the engineered bacteria to produce shikimic acid.

More specifically, to produce shikimic acid in an economical manner, an E. coli strain that is otherwise wild-type, for example, MG1655 or W3110, may be engineered to have the following alterations:
1. The chromosomal copies of aroK and aroL genes are deleted or otherwise mutated.
2. The chromosomal copy of the ptsI gene is optionally deleted or otherwise mutated.
3. The glf gene of Zymomonas mobilis is constitutively expressed.
4. The chromosomal copy of the pyruvate kinase gene is optionally deleted.
5. The following gene fusions are constituted into an operon and expressed from a regulated promoter: aroK-ssrA, and optionally ptsI-ssrA, pyruvate kinase-ssrA. The operon is generated by total gene synthesis from a commercial supplier, such as DNA 2.0, Mr. Gene, Blue Heron Biotechnologies, or Genscript. The operon is integrated into the E. coli chromosome.
6. The following regulated promoter systems may be utilized:
   a. The bacteriophage lambda P_{R} promoter, in the presence of a single copy of the c1857 temperature-sensitive allele of the lambda repressor transcribed from a constitutive promoter.
   b. The lactose operon promoter, in the presence of a single copy of the lacI repressor gene transcribed from a constitutive promoter.
   c. A luxR-responsive promoter, in the presence of a gene encoding the LuxR protein.
7. The strain is optionally engineered to express a sucrose transport system and an invertase.

During the growth phase, the strain is grown in a minimal medium such as M9 medium with glucose, sucrose, or molasses as a carbon source, and in the absence of tryptophan, tyrosine, or phenylalanine. When the lambda P_{R} system is used, the strain is grown at 42° C. Upon switching to the production phase, the temperature is lowered to 30° C., whereupon shikimic acid is produced. Without wishing to be bound by theory, upon the shift to 30° C., the genes encoding shikimate kinase, pyruvate kinase, and the phosphotransferase I protein are repressed, and the corresponding proteins are degraded and not replaced, since mRNAs in E. coli are generally unstable and have a half-life of only a few minutes. The cessation of aromatic amino acid synthesis leads to an up-regulation of the initial steps of this pathway, such as the genes aroF, aroG, and aroH, which encode DAHP synthases. The loss of pyruvate kinase activity leads to an accumulation of phosphoenolpyruvate (PEP), one of the substrates of DAHP synthase. The loss of the phosphotransferase I protein leads to a cessation of glucose transport by the phosphotransferase system, further assisting in PEP accumulation. The loss of shikimate kinase activity results in accumulation of shikimic acid, which is collected by standard procedures.

The E. coli strain described above optionally includes other modifications described by Kraemer et al. (op. cit.), including but not limited to deletion of the shikimate transporter shiA, and use of an AroD/E-homologous protein from N. tabacum to reduce production of quinic acid.

It should be noted that the extent of repression of the various genes is determined by routine experimentation. For example, it is sometimes useful to separately regulate pyruvate kinase so that its activity is reduced but not completely abolished, so that the citric acid cycle may operate and some ATP may be produced by oxidative phosphorylation. Alternatively, pyruvate kinase may be left unmutated.

### Production of Fatty Acids and Alcohols

Biofuels often derive from fatty acids that are derivatized into esters or reduced to fatty alcohols. The starting point for fatty acid synthesis is acetyl-CoA, which is also the starting point for the tricarboxylic acid cycle. According to the invention, it is useful to construct a gene encoding a fusion protein that includes citrate synthase and ssrA, expressed from a regulated promoter. Such a construction has the effect of preventing entry into the TCA cycle, with the result that acetyl-CoA is preferentially directed into fatty acid synthesis. Depending on which other metabolic engineering has been performed, production of ethanol may be enhanced.

As an alternative strategy to producing fatty acids, instead of amino acids, it is sometimes useful to block the synthesis of aromatic amino acids by blocking DAHP synthase. This has the effect of preventing new protein synthesis, leading to some accumulation of other amino acids and feedback inhibition of the enzymes that initiate pathways for their synthesis. Accordingly, a DAHP synthase-ssrA fusion protein is expressed from a regulated promoter, and the promoter is turned off when production of a fatty acid product or related product is desired. In the specific case of E. coli, three isotypes of DAHP synthase are encoded by the genes aroF, aroG, and aroH. To apply this method of the invention to E. coli, it is generally useful to inactivate the chromosomal copies of these genes by mutation, then construct a fusion of one of genes to DNA encoding the ssrA peptide, which is then placed under the control of a regulated promoter.

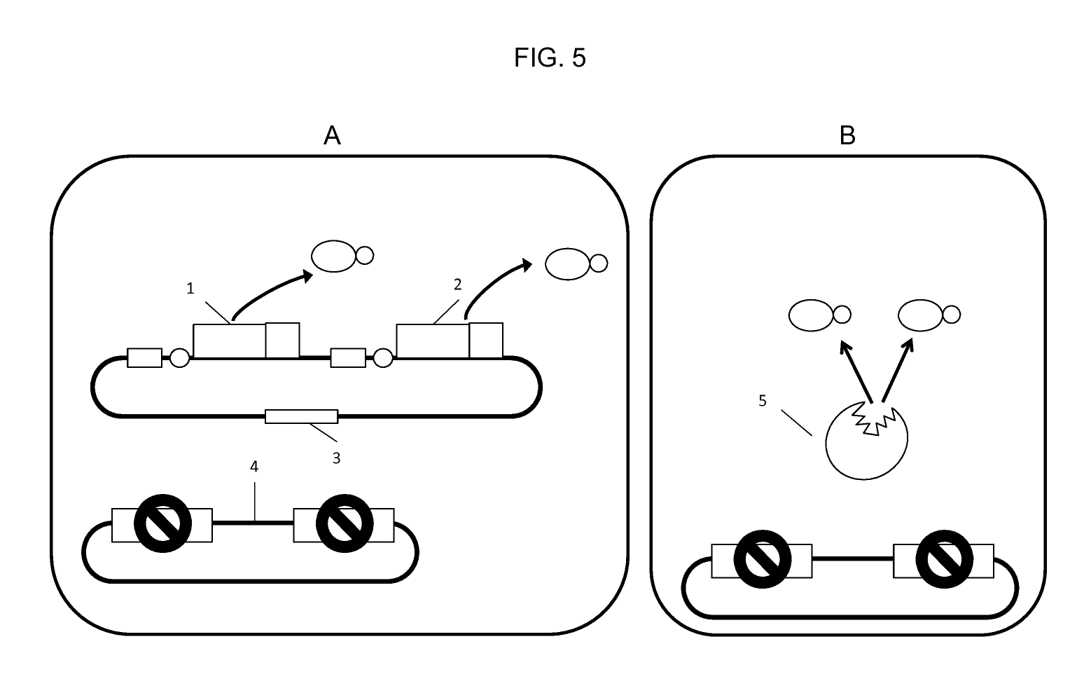

As a first illustration, consider the synthesis of dodecanoic acid (lauric acid; C12 fatty acid; CH₃(CH₂)₁₀COOH). Voelker and Davies (J. Bact. [1994] 176[23]7320-7327) described an engineered E. coli that expressed a plant C12 thioesterase and also carried a knockout of the fadD. The C12 thioesterase has the effect of releasing lauric acid from acyl carrier protein during fatty acid synthesis, and the fadD encodes a fatty acid degradation enzyme that recycles the carbon in fatty acids that cannot be incorporated into membranes. The C 12 thioesterase-expressing fadD knockout strain synthesizes lauric acid at a high level. However, it is noteworthy that this strain grows and divides (FIG. 5 of Voelker and Davies), evidently converting much of the input carbon into biomass even though the C12 thioesterase is expressed constitutively at a high level. According to the invention, when a C12 thioesterase-expressing fadD knockout strain is also engineered to express a DAHP synthase-ssrA fusion protein from a regulated promoter, and the promoter is turned off, the DAHP synthase-ssrA fusion protein is degraded and not replaced, protein synthesis essentially ceases, and production of lauric acid is enhanced relative to the C12 thioesterase-expressing fadD knockout strain.

As a second illustration, consider the synthesis of isobutanol ((CH₃)₂CHCH₂OH). Atsumi et al. (Nature [3 Jan. 2008] 451:86-90) described an engineered E. coli that expressed an artificial operon that expressed high levels of isobutanol by a combination of valine biosynthesis genes, 2-ketoacid decarboxylase, and alcohol dehydrogenase. According to the invention, when a strain expressing valine synthesis genes, 2-ketoacid decarboxylase, and alcohol dehydrogenase is also engineered to express a DAHP synthase-ssrA fusion protein from a regulated promoter, and the promoter is turned off, the DAHP synthase-ssrA fusion protein is degraded and not replaced, protein synthesis essentially ceases, and production of isobutanol is enhanced relative to the parental isobutanol-secreting strain.

More broadly, Atsumi et al. described the production of a variety of alpha-keto carboxylic acids such as 2-ketobutyrate, 2-ketoisovalerate, 2-ketovalerate, 2-keto-3-methyl-valerate, 2-keto-4-methyl-valerate, and phenylpyruvate, which can be decarboxylated to create an aldehyde and then reduced by the serial actions of 2-ketoacid decarboxylase, and alcohol dehydrogenase, to create a series of useful alcohols. According to the invention, when such strains are also engineered to express a DAHP synthase-ssrA fusion protein from a regulated promoter, and the promoter is turned off, the DAHP synthase-ssrA fusion protein is degraded and not replaced, protein synthesis essentially ceases, and production of the desired alcohols is enhanced relative to the parental alcohol-producing strains.

### Sequences Provided by the Invention

The following protein and DNA sequences further illustrate the invention.

Shikimate kinase (AroK)-ssrA
(SEQ ID NO: 14)
MAEKRNIFLVGPMGAGKSTIGRQLAQQLNMEFYDSDQEIEKRTGADVGWVF
DLEGEEGFRDREEKVINELTEKQGIVLATGGGSVKSRETRNRLSARGVVVYLE
TTIEKQLARTQRDKKRPLLHVETPPREVLEALANERNPLYEEIADVTIRTDDQS
AKVVANQIIHMLESNAANDENYALAA
Shikimate kinase-linker-ssrA
(SEQ ID NO: 15)
MAEKRNIFLVGPMGAGKSTIGRQLAQQLNMEFYDSDQEIEKRTGADVGWVF
DLEGEEGFRDREEKVINELTEKQGIVLATGGGSVKSRETRNRLSARGVVVYLE
TTIEKQLARTQRDKKRPLLHVETPPREVLEALANERNPLYEEIADVTIRTDDQS
AKVVANQIIHMLESNGGSGGAANDENYALAA
λO-Shikimate kinase
(SEQ ID NO: 16)
MTNTAKILNFGRASMAEKRNIFLVGPMGAGKSTIGRQLAQQLNMEFYDSDQE
IEKRTGADVGWVFDLEGEEGFRDREEKVINELTEKQGIVLATGGGSVKSRETR
NRLSARGVVVYLETTIEKQLARTQRDKKRPLLHVETPPREVLEALANERNPL
YEEIADVTIRTDDQSAKVVANQIIHMLESN
λO-linker-Shikimate kinase
(SEQ ID NO: 17)
MTNTAKILNFGRASGGSGGMAEKRNIFLVGPMGAGKSTIGRQLAQQLNMEF
YDSDQEIEKRTGADVGWVFDLEGEEGFRDREEKVINELTEKQGIVLATGGGS
VKSRETRNRLSARGVVVYLETTIEKQLARTQRDKKRPLLHVETPPREVLEALA
NERNPLYEEIADVTIRTDDQSAKVVANQIIHMLESN
PtsI-ssrA
(SEQ ID NO: 18)
MISGILASPGIAFGKALLLKEDEIVIDRKKISADQVDQEVERFLSGRAKASAQL
ETIKTKAGETFGEEKEAIFEGHIMLLEDEELEQEIIALIKDKHMTADAAAHEVI
EGQASALEELDDEYLKERAADVRDIGKRLLRNILGLKIIDLSAIQDEVILVAAD
LTPSETAQLNLKKVLGFITDAGGRTSHTSIMARSLELPAIVGTGSVTSQVKND
DYLILDAVNNQVYVNPTNEVIDKMRAVQEQVASEKAELAKLKDLPAITLDG
HQVEVCANIGTVRDVEGAERNGAEGVGLYRTEFLEMDRDALPTEEEQFAAY
KAVAEACGSQAVIVRTMDIGGDKELPYMNFPKEENPFLGWRAIRIAMDRREI
LRDQLRAILRASAFGKLRIMFPMIISVEEVRALRKEIEIYKQELRDEGKAF
DESIEIGVMVETPAAATIARHLAKEVDFFSIGTNDLTQYTLAVDRGNDMISHL
YQPMSPSVLNLIKQVIDASHAEGKWTGMCGELAGDERATLLLLGMGLDEFS
MSAISIPRIKKIIRNTNFEDAKVLAEQALAQPTTDELMTLVNKFIEEKTICAAND
ENYALAA
Pyruvate kinase I-ssrA (pykF as opposed to pykA)
(SEQ ID NO: 19)
MKKTKIVCTIGPKTESEEMLAKMLDAGMNVMRLNFSHGDYAEHGQRIQNLR
NVMSKTGKTAAILLDTKGPEIRTMKLEGGNDVSLKAGQTFTFTTDKSVIGNSE
MVAVTYEGFTTDLSVGNTVLVDDGLIGMEVTAIEGNKVICKVLNNGDLGEN
KGVNLPGVSIALPALAEKDKQDLIFGCEQGVDFVAASFIRKRSDVIEIREHLKA
HGGENIHIISKIENQEGLNNFDEILEASDGIMVARGDLGVEIPVEEVIFAQKMMI
EKCIRARKVVITATQMLDSMIKNPRPTRAEAGDVANAILDGTDAVMLSGESA
KGKYPLEAVSIMATICERTDRVMNSRLEFNNDNRKLRITEAVCRGAVETAEK
LDAPLIVVATQGGKSARAVRKYFPDATILALTTNEKTAHQLVLSKGVVPQLV
KEITSTDDFYRLGKELALQSGLAHKGDVVVMVSGALVPSGTTNTASVHVLA
ANDENYALAA
Citrate synthase-ssrA (gltA)
(SEQ ID NO: 20)
MADTKAKLTLNGDTAVELDVLKGTLGQDVIDIRTLGSKGVFTFDPGFTSTAS
CESKITFIDGDEGILLHRGFPIDQLATDSNYLEVCYILLNGEKPTQEQYDEFKTT
VTRHTMIHEQITRLFHAFRRDSHPMAVMCGITGALAAFYHDSLDVNNPRHRE
IAAFRLLSKMPTMAAMCYKYSIGQPFVYPRNDLSYAGNFLNMMFSTPCEPYE
VNPILERAMDRILILHADHEQNASTSTVRTAGSSGANPFACIAAGIASLWGPA
HGGANEAALKMLEEISSVKHIPEFVRRAKDKNDSFRLMGFGHRVYKNYDPR
ATVMRETCHEVLKELGTKDDLLEVAMELENIALNDPYFIEKKLYPNVDFYSGI
ILKAMGIPSSMFTVIFAMARTVGWIAHWSEMHSDGMKIARPRQLYTGYEKRD
FKSDIKRAANDENYALAA
DAHP-ssrA (tyrosine-repressible)
(SEQ ID NO: 21)
MQKDALNNVHITDEQVLMTPEQLKAAFPLSLQQEAQIADSRKSISDIIAGRDP
RLLVVCGPCSIHDPETALEYARRFKALAAEVSDSLYLVMRVYFEKPRTTVGW
KGLINDPHMDGSFDVEAGLQIARKLLLELVNMGLPLATEALDPNSPQYLGDL
FSWSAIGARTTESQTHREMASGLSMPVGFKNGTDGSLATAINAMRAAAQPHR
FVGINQAGQVALLQTQGNPDGHVILRGGKAPNYSPADVAQCEKEMEQAGLR
PSLMVDCSHGNSNKDYRRQPAVAESVVAQIKDGNRSIIGLMIESNIHEGNQSS
EQPRSEMKYGVSVTDACISWEMTDALLREIHQDLNGQLTARVAAANDENYA
LAA
ClpX (unfoldase from E. coli)
(SEQ ID NO: 22)
MTDKRKDGSGKLLYCSFCGKSQHEVRKLIAGPSVYICDECVDLCNDIIREEIK
EVAPHRERSALPTPHEIRNHLDDYVIGQEQAKKVLAVAVYNHYKRLRNGDTS
NGVELGKSNILLIGPTGSGKTLLAETLARLLDVPFTMADATTLTEAGYVGEDV
ENIIQKLLQKCDYDVQKAQRGIVYIDEIDKISRKSDNPSITRDVSGEGVQQALL
KLIEGTVAAVPPQGGRKHPQQEFLQVDTSKILFICGGAFAGLDKVISHRVETG
SGIGFGATVKAKSDKASEGELLAQVEPEDLIKFGLIPEFIGRLPVVATLNELSEE
ALIQILKEPKNALTKQYQALFNLEGVDLEFRDEALDAIAKKAMARKTGARGL
RSIVEAALLDTMYDLPSMEDVEKVVIDESVIDGQSKPLLIYGKPEAQQASGE
ClpA (unfoldase from E. coli)
(SEQ ID NO: 23)
MLNQELELSLNMAFARAREHRHEFMTVEHLLLALLSNPSAREALEACSVDLV
ALRQELEAFIEQTTPVLPASEEERDTQPTLSFQRVLQRAVFHVQSSGRNEVTG
ANVLVAIFSEQESQAAYLLRKHEVSRLDVVNFISHGTRKDEPTQSSDPGSQPN
SEEQAGGEERMENFTTNLNQLARVGGIDPLIGREKELERAIQVLCRRRKNNPL
LVGESGVGKTAIAEGLAWRIVQGDVPEVMADCTIYSLDIGSLLAGTKYRGDF
EKRFKALLKQLEQDTNSILFIDEIHTIIGAGAASGGQVDAANLIKPLLSSGKIRV
IGSTTYQEFSNIFEKDRALARRFQKIDITEPSIEETVQIINGLKPKYEAHHDVRY
TAKAVRAAVELAVKYINDRHLPDKAIDVIDEAGARARLMPVSKRKKTVNVA
DIESVVARIARIPEKSVSQSDRDTLKNLGDRLKMLVFGQDKAIEALTEAIKMA
RAGLGHEHKPVGSFLFAGPTGVGKTEVTVQLSKALGIELLRFDMSEYMERHT
VSRLIGAPPGYVGFDQGGLLTDAVIKHPHAVLLLDEIEKAHPDVFNILLQVMD
NGTLTDNNGRKADFRNVVLVMTTNAGVRETERKSIGLIHQDNSTDAMEEIKK
IFTPEFRNRLDNIIWFDHLSTDVIHQVVDKFIVELQVQLDQKGVSLEVSQEARN
WLAEKGYDRAMGARPMARVIQDNLKKPLANELLFGSLVDGGQVTVALDKE
KNELTYGFQSAQKHKAEAAH
ClpP (protease from E. coli)
(SEQ ID NO: 24)
MSYSGERDNFAPHMALVPMVIEQTSRGERSFDIYSRLLKERVIFLTGQVEDHM
ANLIVAQMLFLEAENPEKDIYLYINSPGGVITAGMSIYDTMQFIKPDVSTICMG
QAASMGAFLLTAGAKGKRFCLPNSRVMIHQPLGGYQGQATDIEIHAREILKV
KGRMNELMALHTGQSLEQIERDTERDRFLSAPEAVEYGLVDSILTHRN
SspB (adaptor from E. coli)
(SEQ ID NO: 25)
MDLSQLTPRRPYLLRAFYEWLLDNQLTPHLVVDVTLPGVQVPMEYARDGQI
VLNIAPRAVGNLELANDEVRFNARFGGIPRQVSVPLAAVLAIYARENGAGTM
FEPEAAYDEDTSIMNDEEASADNETVMSVIDGDKPDH
DDDTHPDDEPPQPPRGGRPALRVVK
ClpS (N-end rule adaptor from E. coli)
(SEQ ID NO: 26)
MGKTNDWLDFDQLAEEKVRDALKPPSMYKVILVNDDYTPMEFVIDVLQKFF
SYDVERATQLMLAVHYQGKAICGVFTAEVAETKVAMVNKYARENEHPLLCT
LEKA
ccSspB (adaptor from C. crescentus)
(SEQ ID NO: 27)
MSQTEPPEDLMQYEAMAQDALRGVVKAALKKAAAPGGLPEPHHLYITFKTK
AAGVSGPQDLLSKYPDEMTIVLQHQYWDLAPGETFFSVTLKFGGQPKRLSVP
YAALTRFYDPSVQFALQFSAPEIIEDEPEPDPEPEDK
ANQGASGDEGPKIVSLDQFRKK
GBW168-aroK locus (insertion shown in lower-case font)
(SEQ ID NO: 28)
GAAGTTCTGGAAGCGTTGGCCAATGAACGCAATCCGCTGTATGAAGAGAT
TGCCGACGTGACCATTCGTACTGATGATCAAAGCGCTAAAGTGGTTGCAA
ACCAGATTATTCACATGCTGGAAAGCAACgcagctaacgatgaaaactacagcgaaaactatg
ctgacgctagctaatactagagctgatccttcaactcagcaaaagttcgatttattcaacaaagccacgttgtgtctcaaaatct
ctgatgttacattgcacaagataaaaatatatcatcatgaacaataaaactgtctgcttacataaacagtaatacaaggggtgtt
atgagccatattcaacgggaaacgtcttgctcccgtccgcgcttaaactccaacatggacgctgatttatatgggtataaatg
ggctcgcgataatgtcgggcaatcaggtgcgacaatctatcgcttgtatgggaagcccgatgcgccagagttgtttctgaaa
catggcaaaggtagcgttgccaatgatgttacagatgagatggtccgtctcaactggctgacggagtttatgcctacccga
ccatcaagcattttatccgtactcctgatgatgcgtggttactcaccaccgcgattcctgggaaaacagccttccaggtattag
aagaatatcctgattcaggtgaaaatattgttgatgcgctggccgtgttcctgcgccggttacattcgattcctgtttgtaattgt
ccttttaacagcgatcgtgtatttcgtcttgctcaggcgcaatcacgcatgaataacggtttggttgatgcgagtgattttgatga
cgagcgtaatggctggcctgttgaacaagtctggaaagaaatgcacaagctcttgccattctcaccggattcagtcgtcact
catggtgatttctcacttgataaccttatttttgacgaggggaaattaataggttgtattgatgttggacgggtcggaatcgcag
accgttaccaggaccttgccattctttggaactgcctcggtgagttttctccttcattacagaaacggattttcaaaaatatggt
attgataatcctgatatgaataaattgcagtttcatttgatgctcgatgagtttttctaataaTTCTGGCTTTATATA
CACTCGTCTGCGGGTACAGTAATTAAGGTGGATGTCGCGTTATGGAGAGG
ATTGTCGTTACTCTCGGGGAACGTAGTTACCCAAT
Xba-B0032-TACTAG-AroKfwd
(SEQ ID NO: 29)
ggccgcttctagagtcacacaggaaagtactagatggcagagaaacgcaatatctttc
AroK-LAA-spe-pstrev
(SEQ ID NO: 30)
ggccctgcagcggccgctactagtattaagcagccagagcataattttcatcgttagagcgttgctttccagcatgtgaataa
tc
pSB3C5
(SEQ ID NO: 31)
tactagtagcggccgctgcaggagtcactaagggttagttagttagattagcagaaagtcaaaagcctccgaccggaggct
tttgactaaaacttcccttggggttatcattggggctcactcaaaggcggtaatcagataaaaaaaatccttagctttcgctaag
gatgatttctgctagagatggaatagactggatggaggcggataaagttgcaggaccacttctgcgctcggcccttccggct
ggctggtttattgctgataaatctggagccggtgagcgtgggtctcgcggtatcattgcagcactggggccagatggtaagc
cctcccgtatcgtagttatctacacgacggggagtcaggcaactatggatgaacgaaatagacagatcgctgagataggtg
cctcactgattaagcattggtaactgtcagaccaagtttactcatatatactttagattgatttaaaacttcatttttaatttaaa
aggatctaggtgaagatcctttttgataatctcatgaccaaaatcccttaacgtgagttttcgttccactgagcgtcagacccctt
aataagatgatcttcttgagatcgttttggtctgcgcgtaatctcttgctctgaaaacgaaaaaaccgccttgcagggcggttttt
cgaaggttctctgagctaccaactctttgaaccgaggtaactggcttggaggagcgcagtcaccaaaacttgtcctttcagtttag
ccttaaccggcgcatgacttcaagactaactcctctaaatcaattaccagtggctgctgccagtggtgcttttgcatgtctttcc
gggttggactcaagacgatagttaccggataaggcgcagcggtcggactgaacggggggttcgtgcatacagtccagctt
ggagcgaactgcctacccggaactgagtgtcaggcgtggaatgagacaaacgcggccataacagcggaatgacaccgg
taaaccgaaaggcaggaacaggagagcgcacgagggagccgccaggggaaacgcctggtatctttatagtcctgtcgg
gtttcgccaccactgatttgagcgtcagatttcgtgatgcttgtcaggggggcggagcctatggaaaaacggctttgccgcg
gccctctcacttccctgttaagtatcttcctggcatcttccaggaaatctccgccccgttcgtaagccatttccgctcgccgcag
tcgaacgaccgagcgtagcgagtcagtgagcgaggaagcggaatatatcctgtatcacatattctgctgacgcaccggtg
cagccttttttctcctgccacatgaagcacttcactgacaccctcatcagtgccaacatagtaagccagtatacactccgctag
cgctgaggtctgcctcgtgaagaaggtgttgctgactcataccaggcctgaatcgccccatcatccagccagaaagtgagg
gagccacggttgatgagagctttgttgtaggtggaccagttggtgattttgaacttttgctttgccacggaacggtctgcgttgt
cgggaagatgcgtgatctgatccttcaactcagcaaaagttcgatttattcaacaaagccacgttgtgtctcaaaatctctgat
gttacattgcacaagataaaaatatatcatcatgaacaataaaactgtctgcttacataaacagtaatacaaggggtgtttacta
gaggttgatcgggcacgtaagaggttccaactttcaccataatgaaataagatcactaccgggcgtattttttgagttatcgag
attttcaggagctaaggaagctaaaatggagaaaaaaatcacgggatataccaccgttgatatatcccaatggcatcgtaaa
gaacattttgaggcatttcagtcagttgctcaatgtacctataaccagaccgttcagctggatattacggcctttttaaagaccg
taaagaaaaataagcacaagttttatccggcctttattcacattcttgcccgcctgatgaacgctcacccggagtttcgtatgg
ccatgaaagacggtgagctggtgatctgggatagtgttcacccttgttacaccgttttccatgagcaaactgaaacgttttcgt
ccctctggagtgaataccacgacgatttccggcagtttctccacatatattcgcaagatgtggcgtgttacggtgaaaacctg
gcctatttccctaaagggtttattgagaatatgttttttgtctcagccaatccctgggtgagtttcaccagttttgatttaaacgt
ggccaatatggacaacttcttcgcccccgttttcacgatgggcaaatattatacgcaaggcgacaaggtgctgatgccgctggc
gatccaggttcatcatgccgtttgtgatggatccatgtcggccgcatgcttaatgaattacaacagtactgtgatgagtggca
gggcggggcgtaataatactagctccggcaaaaaaacgggcaaggtgtcaccaccctgccctttttctttaaaaccgaaaa
gattacttcgcgtttgccacctgacgtctaagaaaaggaatattcagcaatttgcccgtgccgaagaaaggcccacccgtga
aggtgagccagtgagttgattgctacgtaattagttagttagcccttagtgactcgaattcgcggccgcttctagag
Bba_F2620
(SEQ ID NO: 32)
tccctatcagtgatagagattgacatccctatcagtgatagagatactgagcactactagagaaagaggagaaatactagat
gaaaaacataaatgccgacgacacatacagaataattaataaaattaaagcttgtagaagcaataatgatattaatcaatgctt
atctgatatgactaaaatggtacattgtgaatattatttactcgcgatcatttatcctcattctatggttaaatctgatatttcaa
tcctagataattaccctaaaaaatggaggcaatattatgatgacgctaatttaataaaatatgatcctatagtagattattctaac
tccaatcattcaccaattaattggaatatatttgaaaacaatgctgtaaataaaaaatctccaaatgtaattaaagaagcgaaaac
atcaggtcttatcactgggtttagtttccctattcatacggctaacaatggcttcggaatgcttagttttgcacattcagaaaaag
acaactatatagatagtttatttttacatgcgtgtatgaacataccattaattgttccttctctagttgataattatcgaaaaata
aatatagcaaataataaatcaaacaacgatttaaccaaaagagaaaaagaatgtttagcgtgggcatgcgaaggaaaaagctcttg
ggatatttcaaaaatattaggttgcagtgagcgtactgtcactttccatttaaccaatgcgcaaatgaaactcaatacaacaaacc
gctgccaaagtatttctaaagcaattttaacaggagcaattgattgcccatactttaaaaattaataacactgatagtgctagtgt
agatcactactagagccaggcatcaaataaaacgaaaggctcagtcgaaagactgggcctttcgttttatctgttgtttgtcggt
gaacgctctctactagagtcacactggctcaccttcgggtgggcctttctgcgtttatatactagagacctgtaggatcgtaca
ggtttacgcaagaaaatggtttgttatagtcgaataaa
Bba_R0010
(SEQ ID NO: 33)
caatacgcaaaccgcctctccccgcgcgttggccgattcattaatgcagctggcacgacaggtttcccgactggaaagcg
ggcagtgagcgcaacgcaattaatgtgagttagctcactcattaggcaccccaggctttacactttatgcttccggctcgtatg
ttgtgtggaattgtgagcggataacaatttcacaca
Ribosome binding site (Bba_B0032)
(SEQ ID NO: 34)
tcacacaggaaag
aroK (open reading frame)
(SEQ ID NO: 35)
atggcagagaaacgcaatatctttctggttgggcctatgggtgccggaaaaagcactattgggcgccagttagctcaacaa
ctcaatatggaattttacgattccgatcaagagattgagaaacgaaccggagctgatgtgggctgggttttcgatttagaagg
cgaagaaggcttccgcgatcgcgaagaaaaggtcatcaatgagttgaccgagaaacagggtattgtgctggctactggcg
gcggctctgtgaaatCccgtgaaacgcgtaaccgtCTttccgctcgtggcgttgtcgtttatcttgaaacgaccatcgaaaag
caacttgcacgcacgcagcgtgataaaaaacgcccgttgctgcacgttgaaacaccgccgcgtgaagttctggaagcgtt
ggccaatgaacgcaatccgctgtatgaagagattgccgacgtgaccattcgtactgatgatcaaagcgctaaagtggttgc
aaaccagattattcacatgctggaaagcaac
sspB (open reading frame)
(SEQ ID NO: 36)
atggatttgtcacagctaacaccacgtcgtccctatctgctgcgtgcattctatgagtggttgctggataaccagctcacgccg
cacctggtggtggatgtgacgctccctggcgtgcaggttcctatggaatatgcgcgtgacgggcaaatcgtactcaacattg
cgccgcgtgctgtcggcaatctggaactggcgaatgatgaggtgcgctttaacgcgcgctttggtggcattccgcgtcagg
tttctgtgccgctggctgccgtgctggctatctacgcccgtgaaaatggcgcaggcacgatgtttgagcctgaagctgccta
cgatgaagataccagcatcatgaatgatgaagaggcatcggcagacaacgaaaccgttatgtcggttattgatggcgacaa
gccagatcacgatgatgacactcatcctgacgatgaacctccgcagccaccacgcggtggtcgaccggcattacgcgttg
tgaagtaa
Nucleic acid sequence for AANDENYALAA
(SEQ ID NO: 37)
gcagctaacgatgaaaattatgctctggctgcttaa
Nucleic acid sequence for AANDENYALVA
(SEQ ID NO: 38)
gcagctaacgatgaaaattatgctctggttgcttaa
Nucleic acid sequence for AANDENYADAS
(SEQ ID NO: 39)
gcagctaacgatgaaaattatgctgacgctagctaa
Nucleic acid sequence for AANDENYALDD
(SEQ ID NO: 40)
gcagctaacgatgaaaattatgactggacgactaa
Representative assembled construct
F2620-B0032-AroK-LVA-pSB3C5 (circular)
(SEQ ID NO: 41)
gaattcgcggccgcttctagtccctatcagtgatagagattgacatccctatcagtgatagagatactgagcactactagaga
aagaggagaaatactagatgaaaaacataaatgccgacgacacatacagaataattaataaaattaaagcttgtagaagca
ataatgatattaatcaatgcttatctgatatgactaaaatggtacattgtgaatattatttactcgcgatcatttatcctcattct
atggttaaatctgatatttcaatcctagataattaccctaaaaaatggaggcaatattatgatgacgctaatttaataaaatatga
tcctatagtagattattctaactccaatcattcaccaattaattggaatatatttgaaaacaatgctgtaaataaaaaatctccaa
atgtaattaaagaagcgaaaacatcaggtcttatcactgggtttagtttccctattcatacggctaacaatggcttcggaatgctt
agttttgcacattcagaaaaagacaactatatagatagtttatttttacatgcgtgtatgaacataccattaattgttccttctct
agttgataattatcgaaaaataaatatagcaaataataaatcaaacaacgatttaaccaaaagagaaaaagaatgtttagcgtggg
catgcgaaggaaaaagctcttgggatatttcaaaaatattaggttgcagtgagcgtactgtcactttccatttaaccaatgcgcaa
atgaaactcaatacaacaaaccgctgccaaagtatttctaaagcaattttaacaggagcaattgattgcccatactttaaaaatta
ataacactgatagtgctagtgtagatcactactagagccaggcatcaaataaaacgaaaggctcagtcgaaagactgggcc
tttcgttttatctgttgtttgtcggtgaacgctctctactagagtcacactggctcaccttcgggtgggcctttctgcgtttatat
actagagacctgtaggatcgtacaggtttacgcaagaaaatggtttgttatagtcgaataaatactagagtcacacaggaaagta
ctagatggcagagaaacgcaatatctttctggttgggcctatgggtgccggaaaaagcactattgggcgccagttagctcaa
caactcaatatggaattttacgattccgatcaagagattgagaaacgaaccggagctgatgtgggctgggttttcgatttaga
aggcgaagaaggcttccgcgatcgcgaagaaaaggtcatcaatgagttgaccgagaaacagggtattgtgctggctactg
gcggcggctctgtgaaatcccgtgaaacgcgtaaccgtctttccgctcgtggcgttgtcgtttatcttgaaacgaccatcgaa
aagcaacttgcacgcacgcagcgtgataaaaaacgcccgttgctgcacgttgaaacaccgccgcgtgaagttctggaagc
gttggccaatgaacgcaatccgctgtatgaagagattgccgacgtgaccattcgtactgatgatcaaagcgctaaagtggtt
gcaaaccagattattcacatgctggaaagcaacgcagctaacgatgaaaattatgactggttgcttaatactagtagcggcc
gctgcaggagtcactaagggttagttagttagattagcagaaagtcaaaagcctccgaccggaggcttttgactaaaacttc
ccttggggttatcattggggctcactcaaaggcggtaatcagataaaaaaaatccttagctttcgctaaggatgatttctgcta
gagatggaatagactggatggaggcggataaagttgcaggaccacttctgcgctcggcccttccggctggctggtttattg
ctgataaatctggagccggtgagcgtgggtctcgcggtatcattgcagcactggggccagatggtaagccctcccgtatcg
tagttatctacacgacggggagtcaggcaactatggatgaacgaaatagacagatcgctgagataggtgcctcactgatta
agcattggtaactgtcagaccaagtttactcatatatactttagattgatttaaaacttcatttttaatttaaaaggatctaggtg
aagatcctttttgataatctcatgaccaaaatcccttaacgtgagttttcgttccactgagcgtcagaccccttaataagatgatc
ttcttgagatcgttttggtctgcgcgtaatctcttgctctgaaaacgaaaaaaccgccttgcagggcggtttttcgaaggttctct
gagctaccaactctttgaaccgaggtaactggcttggaggagcgcagtcaccaaaacttgtcctttcagtttagccttaaccggc
gcatgacttcaagactaactcctctaaatcaattaccagtggctgctgccagtggtgcttttgcatgtctttccgggttggactc
aagacgatagttaccggataaggcgcagcggteggactgaacggggggttcgtgcatacagtccagcttggagcgaact
gcctacccggaactgagtgtcaggcgtggaatgagacaaacgcggccataacagcggaatgacaccggtaaaccgaaa
ggcaggaacaggagagcgcacgagggagccgccaggggaaacgcctggtatctttatagtcctgtcgggtttcgccacc
actgatttgagcgtcagatttcgtgatgcttgtcaggggggcggagcctatggaaaaacggctttgccgcggccctctcact
tccctgttaagtatcttcctggcatcttccaggaaatctccgccccgttcgtaagccatttccgctcgccgcagtcgaacgacc
gagcgtagcgagtcagtgagcgaggaagcggaatatatcctgtatcacatattctgctgacgcaccggtgcagccttttttct
cctgccacatgaagcacttcactgacaccctcatcagtgccaacatagtaagccagtatacactccgctagcgctgaggtct
gcctcgtgaagaaggtgttgctgactcataccaggcctgaatcgccccatcatccagccagaaagtgagggagccacggt
tgatgagagctttgttgtaggtggaccagttggtgattttgaacttttgctttgccacggaacggtctgcgttgtcgggaagatg
cgtgatctgatccttcaactcagcaaaagttcgatttattcaacaaagccacgttgtgtctcaaaatctctgatgttacattgcac
aagataaaaatatatcatcatgaacaataaaactgtctgcttacataaacagtaatacaaggggtgtttactagaggttgatcg
ggcacgtaagaggttccaactttcaccataatgaaataagatcactaccgggcgtatttttgagttatcgagattttcaggag
ctaaggaagctaaaatggagaaaaaaatcacgggatataccaccgttgatatatcccaatggcatcgtaaagaacattttga
ggcatttcagtcagttgctcaatgtacctataaccagaccgttcagctggatattacggcctttttaaagaccgtaaagaaaaa
taagcacaagttttatccggcctttattcacattcttgcccgcctgatgaacgctcacccggagtttcgtatggccatgaaaga
cggtgagctggtgatctgggatagtgttcacccttgttacaccgttttccatgagcaaactgaaacgttttcgtccctctggagt
gaataccacgacgatttccggcagtttctccacatatattcgcaagatgtggcgtgttacggtgaaaacctggcctatttccct
aaagggtttattgagaatatgttttttgtctcagccaatccctgggtgagtttcaccagttttgatttaaacgtggccaatatgga
caacttatcgcccccgttttcacgatgggcaaatattatacgcaaggcgacaaggtgctgatgccgctggcgatccaggttc
atcatgccgtttgtgatggcttccatgtcggccgcatgcttaatgaattacaacagtactgtgatgagtggcagggcggggc
gtaataatactagctccggcaaaaaaacgggcaaggtgtcaccaccctgccctttttctttaaaaccgaaaagattacttcgc
gtttgccacctgacgtctaagaaaaggaatattcagcaatttgcccgtgccgaagaaaggcccacccgtgaaggtgagcc
agtgagttgattgctacgtaattagttagttagcccttagtgactc
Branched amino acid production (C. glutamicum)
cg-ilvE-AANDENYALVA
(SEQ ID NO: 42)
atgacgtcattagagttcacagtaacccgtaccgaaaatccgacgtcacccgatcgtctgaaggaaattcttgccgcaccga
agttcggtaagttcttcaccgaccacatggtgaccattgactggaacgagtcggaaggctggcacaacgcccaattagtgc
catacgcgccgattcctatggatcctgccaccaccgtattccactacggacaggcaatttttgagggaattaaggcctaccg
ccattcggacgaaaccatcaagactttccgtcctgatgaaaacgccgagcgtatgcagcgttcagcagctcgaatggcaat
gccacagttgccaaccgaggactttattaaagcacttgaactgctggtagacgcggatcaggattgggttcctgagtacggc
ggagaagcttccctctacctgcgcccattcatgatctccaccgaaattggcttgggtgtcagcccagctgatgcctacaagtt
cctggtcatcgcatccccagtcggcgcttacttcaccggtggaatcaagcctgtttccgtctggctgagcgaagattacgtcc
gcgctgcacccggcggaactggtgacgccaaatttgctggcaactacgcggcttctttgcttgcccagtcccaggctgcgg
aaaagggctgtgaccaggtcgtatggttggatgccatcgagcacaagtacatcgaagaaatgggtggcatgaacttggg
ttcatctaccgcaacggcgaccaagtcaagctagtcacccctgaactttccggctcactacttccaggcatcacccgcaagt
cacttctacaagtagcacgcgacttgggatacgaagtagaagagcgaaagatcaccaccaccgagtgggaagaagacg
caaagtctggcgccatgaccgaggcatttgcttgcggtactgcagctgttatcacccctgttggcaccgtgaaatcagctca
cggcaccttcgaagtgaacaacaatgaagtcggagaaatcacgatgaagcttcgtgaaaccctcaccggaattcagcaag
gaaacgttgaagaccaaaacggatggctttacccactggttggcGCAGCTAACGATGAAAATTATGC
TCTGGTGGCTtaa
Branched amino acid production (C. glutamicum)
cg-panB-AANDENYALGG
(SEQ ID NO: 43)
atgtcaggcattgatgcaaagaaaatccgcacccgtcatttccgcgaagctaaagtaaacggccagaaagtttcggttctca
ccagctatgatgcgctttcggcgcgcatttttgatgaggctggcgtcgatatgctccttgttggtgattccgctgccaacgttgt
gctgggtcgcgataccaccttgtcgatcaccttggatgagatgattgtgctggccaaggcggtgacgatcgctacgaagcg
tgcgcttgtggtggttgatctgccgtttggtacctatgaggtgagcccaaatcaggcggtggagtccgcgatccgggtcatg
cgtgaaacgggtgcggctgcggtgaagatcgagggtggcgtggagatcgcgcagacgattcgacgcattgttgatgctg
gaattccggttgtcggccacatcgggtacaccccgcagtccgagcattccttgggcggccacgtggttcagggtcgtggc
gcgagttctggaaagctcatcgccgatgcccgcgcgttggagcaggcgggtgcgtttgcggttgtgttggagatggttcca
gcagaggcagcgcgcgaggttaccgaggatctttccatcaccactatcggaatcggtgccggcaatggcacagatgggc
aggttttggtgtggcaggatgccttcggcctcaaccgcggcaagaagccacgcttcgtccgcgagtacgccaccttgggc
gattccttgcacgacgccgcgcaggcctacatcgccgatatccacgcgggtaccttcccaggcgaagcggagtcctttG
CAGCTAACGATGAAAATTATGCTCTGGGCGGCtaa
Branched amino acid production (C. glutamicum)
cg-leuA-AANDENYALAG
(SEQ ID NO: 44)
atgcttcaccacatgacttcgcgtgcgaatctacttcttcttcgccgcggcgggtcccagaggtctatgtctcctaacgatgca
ttcatctccgcacctgccaagatcgaaaccccagttgggcctcgcaacgaaggccagccagcatggaataagcagcgtg
gctcctcaatgccagttaaccgctacatgcctttcgaggttgaggtagaagatatttctctgccggaccgcacttggccagat
aaaaaaatcaccgttgcacctcagtggtgtgctgttgacctgcgtgacggcaaccaggctctgattgatccgatgtctcctga
gcgtaagcgccgcatgtttgagctgctggttcagatgggcttcaaagaaatcgaggtcggtttcccttcagcttcccagactg
attttgatttcgttcgtgagatcatcgaaaagggcatgatccctgacgatgtcaccattcaggttctggttcaggctcgtgagc
acctgattcgccgtacttttgaagcttgcgaaggcgcaaaaaacgttatcgtgcacttctacaactccacctccatcctgcag
cgcaacgtggtgttccgcatggacaaggtgcaggtgaagaagctggctaccgatgccgctgaactaatcaagaccatcgc
tcaggattacccagacaccaactggcgctggcagtactcccctgagtccttcaccggcactgaggttgagtacgccaagg
aagttgtggacgcagttgttgaggtcatggatccaactcctgagaaccaatgatcatcaacctgccttccaccgttgagatg
atcacccctaacgtttacgcagactccattgaatggatgcaccgcaatctaaaccgtcgtgattccattatcctgtccctgcac
ccgcacaatgaccgtggcaccggcgttggcgcagctgagctgggctacatggctggcgctgaccgcatcgaaggctgc
ctgttcggcaacggcgagcgcaccggcaacgtctgcctggtcaccctggcactgaacatgctgacccagggcgttgacc
ctcagctggacttcaccgatatacgccagatccgcagcaccgttgaatactgcaaccagctgcgcgttcctgagcgccacc
catacggcggtgacctggtcttcaccgctttctccggttcccaccaggacgctgtgaacaagggtctggacgccatggctg
ccaaggttcagccaggtgctagctccactgaagtttcttgggagcagctgcgcgacaccgaatgggaggttccttacctgc
ctatgatccaaaggatgtcggtcgcgactacgaggctgttatccgcgtgaactcccagtccggcaagggcggcgttgctt
acatcatgaagaccgatcacggtctgcagatccctcgctccatgcaggttgagttctccaccgttgtccagaacgtcaccga
cgctgagggcggcgaggtcaactccaaggcaatgtgggatatcttcgccaccgagtacctggagcgcaccgcaccagtt
gagcagatcgcgctgcgcgtcgagaacgctcagaccgaaaacgaggatgcatccatcaccgccgagctcatccacaac
ggcaaggacgtcaccgtcgatggccgcggcaacggcccactggccgcttacgccaacgcgctggagaagctgggcat
cgacgttgagatccaggaatacaaccagcacgcccgcacctcgggcgacgatgcagaagcagccgcctacgtgctggc
tgaggtcaacggccgcaaggtctggggcgtcggcatcgctggctccatcacctacgcttcgctgaaggcagtgacctccg
ccgtaaaccgcgcgctggacgtcaaccacgaggcagtcctggctggcggcgttGCAGCTAACGATGAAA
ATTATGCTCTGGCTGGCtaa
Branched amino acid production (C. glutamicum)
cg-p-ilvE-AANDENYALVA
(SEQ ID NO: 45)
mtsleftvtrtenptspdrlkeilaapkfgkfftdhmvtidwnesegwhnaqlvpyapipmdpattvfhygqaifegik
ayrhsdetiktfrpdenaermqrsaarmampqlptedfikalellvdadqdwypeyggeaslylrpfmisteiglgvsp
adaykflviaspvgayftggikpvsvwlsedyvraapggtgdakfagnyaasllaqsqaaekgcdqvvvvldaiehky
ieemggmnlgfiyrngdqvklvtpelsgsllpgitrksllqvardlgyeveerkittteweedaksgamteafacgtaav
itpvgtvksahgtfevnnnevgeitmklretltgiqqgnvedqngwlyplvgAANDENYALVA
Branched amino acid production (C. glutamicum)
cg-p-panB-AANDENYALGG
(SEQ ID NO: 46)
msgidakkirtrhfreakvngqkvsvltsydalsarifdcagvdmllvgdsaanvvlgrdttlsitldemivlakavtiatk
ralvvvdlpfgtyevspnqavesairvmretgaaavkieggveiaqtirrivdagipvvghigytpqsehslgghvvqg
rgassgkliadaraleqagafavvlemvpaeaarevtedlsittigigagngtdgqvlvwqdafglnrgkkprfvreyatl
gdslhdaaqayiadihagtfpgeaesfAANDENYALGG
Branched amino acid production (C. glutamicum)
cg-p-leuA-AANDENYALAG
(SEQ ID NO: 47)
mlhhmtsranllllrrggsqrsmspndafisapakietpvgprnegqpawnkqrgssmpvnrympfevevedislp
drtwpdkkitvapqwcavdlrdgnqalidpmsperkrrmfellvqmgfkeievgfpsasqtdfdfvreiiekgmipd
dvtiqvlvqarehlirrtfeacegaknvivhfynstsilqrnvvfrmdkvqvkklatdaaeliktiaqdypdtnwrwqys
pesftgteveyakevvdavvevmdptpenpmiinlpstvemitpnvyadsiewmhrnlnrrdsiilslhphndrgtg
vgaaelgymagadriegclfgngertgnvclvtlalnmltqgvdpqldftdirqirstveycnqlrvperhpyggdlvft
afsgshqdavnkgldamaakvqpgasstevsweqlrdtewevpylpidpkdvgrdyeavirvnsqsgkggvayim
ktdhglqiprsmqvefstvvqnvtdaeggevnskamwdifateylertapvegialrvenaqtenedasitaelihngk
dvtvdgrgngplaayanaleklgidveiqeynqhartsgddaeaaayvlaevngrkvwgvgiagsityaslkavtsav
nraldvnheavlaggvAANDENYALAG
Branched amino acid production (E. coli)
ec-ilvE-AANDENYALVA
(SEQ ID NO: 48)
atgaccacgaagaaagctgattacatttggttcaatggggagatggttcgctgggaagacgcgaaggtgcatgtgatgtcg
cacgcgctgcactatggcacttcggtttttgaaggcatccgttgctacgactcgcacaaaggaccggttgtattccgccatcg
tgagcatatgcagcgtctgcatgactccgccaaaatctatcgcttcccggtttcgcagagcattgatgagctgatggaagctt
gtcgtgacgtgatccgcaaaaacaatctcaccagcgcctatatccgtccgctgatcttcgtcggtgatgttggcatgggagta
aacccgccagcgggatactcaaccgacgtgattatcgctgctttcccgtggggagcgtatctgggcgcagaagcgctgga
gcaggggatcgatgcgatggtttcctcctggaaccgcgcagcaccaaacaccatcccgacggcggcaaaagccggtgg
taactacctctcttccctgctggtgggtagcgaagcgcgccgccacggttatcaggaaggtatcgcgctggatgtgaacgg
ttatatctctgaaggcgcaggcgaaaacctgtttgaagtgaaagatggtgtgctgttcaccccaccgttcacctcctccgcgc
tgccgggtattacccgtgatgccatcatcaaactggcgaaagagctgggaattgaagtacgtgagcaggtgctgtcgcgc
gaatccctgtacctggcggatgaagtgtttatgtccggtacggcggcagaaatcacgccagtgcgcagcgtagacggtatt
caggttggcgaaggccgttgtggcccggttaccaaacgcattcagcaagccttcttcggcctcttcactggcgaaaccgaa
gataaatggggctggttagatcaagttaatcaaGCAGCTAACGATGAAAATTATGCTCTGGTG
GCTtaa
Branched amino acid production (E. coli)
ec-panB-AANDENYALGG
(SEQ ID NO: 49)
atgaaaccgaccaccatctccttactgcagaagtacaaacaggaaaaaaaacgtttcgcgaccatcaccgcttatgactata
gcttcgccaaactctttgctgatgaagggcttaacgtcatgctggtgggcgattcgctgggcatgacggttcaggggcacg
actccaccctgccagttaccgttgccgatatcgcctaccacactgccgccgtacgtcgcggcgcaccaaactgcctgctgc
tggctgacctgccgtttatggcgtatgccacgccggaacaagccttcgaaaacgccgcaacggttatgcgtgccggtgcta
acatggtcaaaattgaaggcggtgagtggctggtagaaaccgtacaaatgctgaccgaacgtgccgttcctgtatgtggtc
acttaggtttaacaccacagtcagtgaatattttcggtggctacaaagttcaggggcgcggcgatgaagcgggcgatcaact
gctcagcgatgcattagccttagaagagctggggcacagctgctggtgctggaatgcgtgccggttgaactggcaaaac
gtattaccgaagcactggcgatcccggttattggcattggcgcaggcaacgtcactgacgggcagatcctcgtgatgcacg
acgcctttggtattaccggcggtcacattcctaaattcgctaaaaatttcctcgccgaaacgggcgacatccgcgcggctgt
gcggcagtatatggctgaagtggagtccggcgtttatccgggcgaagaacacagtttccatGCAGCTAACGAT
GAAAATTATGCTCTGGGCGGCtaa
Branched amino acid production (E. coli)
ec-leuA-AANDENYALAG
(SEQ ID NO: 50)
atgagccagcaagtcattattttcgataccacattgcgcgacggtgaacaggcgttacaggcaagcttgagtgtgaaagaa
aaactgcaaattgcgctggcccttgagcgtatgggtgttgacgtgatggaagtcggtttccccgtctcttcgccgggcgatttt
gaatcggtgcaaaccatcgcccgccaggttaaaaacagccgcgtatgtgcgttagctcgctgcgtggaaaaagatatcga
cgtggcggccgaatccctgaaagtcgccgaagccttccgtattcatacctttattgccacttcgccaatgcacatcgccacca
agctgcgcagcacgctggacgaggtgatcgaacgcgctatctatatggtgaaacgcgcccgtaattacaccgatgatgttg
aattttcttgcgaagatgccgggcgtacacccattgccgatctggcgcgagtggtcgaagcggcgattaatgccggtgcca
ccaccatcaacattccggacaccgtgggctacaccatgccgtttgagttcgccggaatcatcagcggcctgtatgaacgcg
tgcctaacatcgacaaagccattataccgtacatacccacgacgatttgggcctggcggtcggaaactcactggcggcgg
tacatgccggtgcacgccaggtggaaggcgcaatgaacgggatcggcgagcgtgccggaaactgttccctggaagaag
tcatcatggcgatcaaagttcgtaaggatattctcaacgtccacaccgccattaatcaccaggagatatggcgcaccagcca
gttagttagccagatttgtaatatgccgatcccggcaaacaaagccattgttggcagcggcgcattcgcacactcctccggt
atacaccaggatggcgtgctgaaaaaccgcgaaaactacgaaatcatgacaccagaatctattggtctgaaccaaatccag
ctgaatctgacctctcgttcggggcgtgcggcggtgaaacatcgcatggatgagatggggtataaagaaagtgaatataatt
tagacaatttgtacgatgcttcctgaagctggcggacaaaaaaggtcaggtgtttgattacgatctggaggcgctggccttc
atcggtaagcagcaagaagagccggagcatttccgtctggattacttcagcgtgcagtctggctctaacgatatcgccacc
gccgccgtcaaactggcctgtggcgaagaagtcaaagcagaagccgccaacggtaacggtccggtcgatgccgtctatc
aggcaattaaccgcatcactgaatataacgtcgaactggtgaaatacagcctgaccgccaaaggccacggtaaagatgcg
ctgggtcaggtggatatcgtcgctaactacaacggtcgccgcttccacggcgtcggcctggctaccgatattgtcgagtcat
ctgccaaagccatggtgcacgttctgaacaatatctggcgtgccgcagaagtcgaaaaagagttgcaacgcaaagctcaa
cacaacgaaaacaacaaggaaaccgtgGCAGCTAACGATGAAAATTATGCTCTGGCTGG
Ctga
Branched amino acid production (E. coli)
ec-p-ilvE-AANDENYALVA
(SEQ ID NO: 51)
mttkkadyiwfngemvrwedakvhvmshalhygtsvfegircydshkgpvvfrhrehmqrlhdsakiyrfpvsqs
idelmeacrdvirknnltsayirplifvgdygmgvnppagystdviiaafpwgaylgaealeqgidamvsswnraap
ntiptaakaggnylssllvgsearrhgyqegialdvngyisegagenlfevkdgvlftppftssalpgitrdaiiklakelgi
evreqvlsreslyladevfmsgtaaeitpvrsvdgiqvgegrcgpvtkriqqaffglftgetedkwgwldqvnqAAN
DENYALVA
Branched amino acid production (E. coli)
ec-p-panB-AANDENYALGG
(SEQ ID NO: 52)
mkpttisllqkykqekkrfatitaydysfaklfadeglnvmlvgdslgmtvqghdstlpvtvadiayhtaavrrgapncl
lladlpfmayatpeqafenaatvmraganrnvkieggewlvetvqmlteravpvcghlgltpqsvnifggykvqgrg
deagdqllsdalaleaagaqllvlecvpvelakritealaipvigigagnvtdgqilvmhdafgitgghipkfaknflaetg
diraavrqymaevesgvypgeehsfhAANDENYALGG
Branched amino acid production (E. coli)
ec-p-leuA-AANDENYALAG
(SEQ ID NO: 53)
msqqviifdttlidgeqalqaslsvkeklqialalermgvdvmevgfpvsspgdfesvqtiarqvknsrvcalarcvek
didvaaeslkvaeafrihtfiatspmhiatklrstldevieraiymvkrarnytddvefscedagrtpiadlarvveaaina
gattinipdtvgytmpfefagiisglyervpnidkaiisvhthddlglavgnslaavhagarqvegamngigeragncsl
eevimaikvrkdilnyhtainhqeiwrtsqlvsqicnmpipankaivgsgafahssgihqdgvlknrenyeimtpesi
glnqiqlnltsrsgraavkhrmdemgykeseynldnlydaflkladkkgqvfdydlealafigkqqeepehfrldyfsv
qsgsndiataavklacgeevkaeaangngpvdavyqainriteynvelvkysltakghgkdalgqvdivanyngrrfh
gvglatdivessakamvhvlnniwraaevekelqrkaqhnennketvAANDENYALAG

### EXAMPLES

The present invention is illustrated by the following examples, which are in no way intended to be limiting of the invention.

### Example 1

### Synthesis of Shikimic Acid from a Microbe Containing an Engineered Shikimate Kinase Gene

An E. coli strain capable of being grown in the absence of aromatic amino acids and producing shikimic acid was engineered as follows. The strain was engineered to express a shikimate kinase isoform, the product of the aroK gene, from a plasmid, while the chromosomal genes encoding shikimate kinase were non-functional. The plasmid-borne shikimate kinase isoform was engineered to have a degradation tag at its C-terminus. In this case and throughout the invention, it was and is useful to inspect the three-dimensional structure of a protein to verify that a chosen terminus is compatible with addition of a degradation tag. The solved structure of the aroK product, PDB file 1KAG, was inspected and the steric availability of the C-terminus was verified.

Plasmid vectors were generated which allow for conditional expression of E. coli shikimate kinase I, aroK. Using standard plasmid construction techniques, the coding sequence for aroK was fused to each of the four degradation tags, AANDENYALAA (SEQ ID NO: 1), AANDENYALVA (SEQ ID NO: 8), AANDENYADAS (SEQ ID NO: 2), and AANDENYALDD (SEQ ID NO: 13). This fusion construct was inserted downstream of either the IPTG-inducible lac promoter (SEQ ID NO: 33) or the HSL-inducible LuxR-derived promoter, F2620 (SEQ ID NO: 32). Each construct contained the ribosome binding site (SEQ ID NO: 34) and resided on the plasmid backbone, pSB3C5 (SEQ ID NO: 31), a chloramphenicol-resistant low-copy plasmid bearing a p15a origin of replication. Nucleotide sequences for each component are listed below, as well as a sample assembled sequence for the construct F2620-B0032-AroK-LVA (SEQ ID NO: 41) as present in pSB3C5.

The complete cloning process for the generation of plasmid F2620-B0032-AroK-LAA (pSB3C5) is described here and the general principles were applied to the generation of the other plasmids. The open reading frame of aroK was PCR amplified from E. coli DH5α chromosomal DNA using primers Xba-B0032-TACTAG-AroKfwd (SEQ ID NO: 29) and AroK-LAA-spe-pstrev (SEQ ID NO: 30) resulting in product PCR1-LAA. F2620 (SEQ ID NO: 32) was generated by PCR resulting in product PCR2-F2620. PCR1-LAA was then incubated with restriction enzymes XbaI and PstI in NEB Buffer #2 supplemented with BSA for 2 hours at 37° C.; PCR2-F2620 was incubated with restriction enzymes EcoRI and SpeI under identical conditions. Successful PCR amplification and restriction digestion was analyzed by gel electrophoresis. After removing heat-denatured restriction enzymes using a Qiagen PCR purification kit, digested PCR1-LAA and PCR2-F2620 were mixed in a stoichiometric ratio with plasmid backbone pSB3C5 which had been treated with EcoRI and PstI. The 3-component mixture was incubated with T4 DNA ligase for 2 hours at room temperature. Chemically competent E. coli NEB 10β cells were then transformed with this ligation product and plated on LB/chloramphenicol. Individual colonies were picked and grown in liquid culture overnight.

Strains of E. coli termed GBW181, GBW182, and GBW183 were engineered as follows. The relevant features were that GBW181, GWB182, and GWB183 contained a version of aroK with a C-terminal “AANDENYADAS” (SEQ ID NO: 2), “AANDENYALVA” (SEQ ID NO: 8), and “AANDENYALDD” (SEQ ID NO: 13), variants of the AANDENYALAA (SEQ ID NO: 1) degradation tag (see table above). Of these, the AANDENYALVA (SEQ ID NO: 8) tag triggered the greatest degradation, while the AANDENYALDD (SEQ ID NO: 13) did not cause degradation and served as a negative control.

In these constructions, the aroK-tag genes were regulated by a strong promoter that was induced by homoserine lactone. Specifically, the aroK gene was expressed from the element F2620 (SEQ ID NO: 32), which encodes a luxR transcriptional regulatory protein that is activated by homoserine lactone (HSL), a LuxR-regulated promoter directing transcription of the E. coli aroK gene fused to a DNA segment encoding AANDENYALVA (SEQ ID NO: 8), and a p15a origin of replication. The chromosomal copies of aroK and aroL were mutated by conventional procedures.

In the following experiments, cells were grown in M9 medium that included 0.4% glucose, 1 μg/ml thiamin, and “tryptophan dropout medium” (Sigma-Aldrich, St. Louis, Mo.), which contains most amino acids but lacks the expensive amino acid tryptophan. This assay system had the advantage that cells would grow more quickly than in a minimal medium without amino acids, while faithfully representing the behavior of cells grown in a minimal medium supplemented only with a carbohydrate source.

The relative degradation-promoting activities of the three different tags were confirmed in a preliminary experiment. Strains 181 and 183 were found to grow in selective medium in the absence of the inducer HSL, while strain 182 only grew in the presence of about 10 nM HSL. These results indicated that low-level expression of the non-induced promoter produced sufficient aroK protein in strains 181 and 183 for tryptophan production, while the aroK protein from strain 182 was too rapidly degraded to allow sufficient tryptophan synthesis for growth.

Cells were inoculated from a single colony and grown with aeration at 37° C. for about 16 hours with 10 nM homoserine lactone to induce the aroK-AANDENYALVA protein. The culture reached an 0D₇₀₀ of about 0.5. At this point, the culture was spun down, resuspended in twice the prior volume, washed in M9 medium without additions, and split into cultures with 10 nM homoserine lactone or with no homoserine lactone, in M9 medium, glucose, thiamin, and tryptophan dropout medium. After about 4 hours, the cultures were spun down and the supernatants were filter-sterilized.

The supernatants were tested for levels of shikimic acid by a bioassay as follows, based on the ability of shikimic acid to support growth of an aroE mutant of E. coli. Each supernatant was diluted 2-fold into fresh medium containing about 10⁴ of an aroK mutant strain of E. coli, JW3242-1 (Coli Genetic Stock Center, New Haven, Conn.). In addition, serial dilutions of shikimic acid were added to similar cultures. The cultures were grown for 24 hours and optical densities compared. Based on this analysis, the shikimic acid level in the culture lacking homoserine lactone was about 10 μg/ml. The culture with 10 nM homoserine lactone produced no detectable shikimic acid.

These results indicated that shikimic acid can be produced from a culture grown in the absence of an aromatic amino acid.

Production of shikimic acid was also observed in a culture of strain 182 grown in the absence of amino acid supplements. A culture is grown in the presence of homoserine lactone in, for example, M9 medium containing glucose, sucrose, glycerol, molasses, or treated cellulosic biomass, is grown to a late logarithmic stage, the homoserine lactone is removed, and shikimic acid is produced by the cells as the aroK product is degraded and not replaced. The resulting shikimic acid is purified from the supernatant. To further improve shikimic acid yields, strain 182 is engineered to express the glf gene from Zymomonas mobilis.

### Example 2

### Production of Shikimic Acid from a Microbial Strain in Which Shikimate Kinase is Fused to a Degradation Tag and Expressed from an Episome with Conditional Replication

In an alternative method of the invention, an E. coli strain that could be grown in the absence of aromatic amino acids and produce shikimic acid was engineered as follows. Four variants were constructed from a plasmid derivative of the low-copy vector pSC101, in which the origin of the plasmid was temperature-sensitive for replication. The plasmid encoded the E. coli aroK gene expressed from its endogenous promoter. The four plasmid variant coding sequences for the degradation tags AANDENYALAA (SEQ ID NO: 1), AANDENYALVA (SEQ ID NO: 8), AANDENYADAS (SEQ ID NO: 2) and the non-degrading control variant AANDENYALDD (SEQ ID NO: 13) were fused to the 3′ end of the aroK coding sequence. These vectors also encoded a chloramphenicol-resistance marker. Expression of shikimate kinase from the E. coli chromosome was defective.

The four strains were inoculated into the M9 glucose thiamin tryptophan-dropout medium described in Example 1 and incubated with aeration at 30° C. for 16 hours. The strains encoding shikimate kinase with the AANDENYADAS (SEQ ID NO: 2) and AANDENYALDD (SEQ ID NO: 13) tags reached near-saturation while the strains encoding shikimate kinase with the AANDENYALAA (SEQ ID NO: 1) and AANDENYALVA (SEQ ID NO: 8) tags showed no detectable growth. The strain encoding the shikimate kinase-AANDENYADAS fusion protein was pelleted in a centrifuge and resuspended in fresh medium for a net 2-fold dilution, and then incubated at 37° C. for about 5.5 hours with aeration. The cells were pelleted in a centrifuge, and the supernatant was withdrawn, filter-sterilized, and tested for shikimic acid levels in the bioassay essentially as described in Example 1. Based on the results of this bioassay, the shikimic acid in the filter-sterilized supernatant of the culture was about 0.05 micrograms/ml.

Without wishing to be bound by theory, shikimic acid was produced by the following mechanism. When the culture bearing plasmid with the shikimate kinase-AANDENYADAS expression construction and the temperature-sensitive origin of replication was transferred to 37° C., replication of the plasmid largely or completely stopped, and the plasmid was lost from many cells during cell division. Once the plasmid was lost from a given cell, the remaining shikimate kinase-AANDENYADAS protein was degraded and not replaced, leaving the cell without shikimate kinase enzyme activity. Such cells produced shikimic acid and secreted this molecule into the medium.

### Other Embodiments

From the foregoing description, it is apparent that variations and modifications may be made to the invention described herein to adopt it to various usages and conditions. Such embodiments are also within the scope of the following claims.

All publications, patent applications, and patents mentioned in this specification are herein incorporated by reference to the same extent as if each independent publication, patent application, or patent was specifically and individually indicated to be incorporated by reference.

What is claimed is:

1. A cell that expresses a metabolic product, said cell comprising a protein, said protein comprising a first moiety with enzymatic activity and a second moiety capable of promoting degradation of said protein, wherein said first and second moieties are not found together in a naturally occurring polypeptide, said cell further comprising a regulatory system, whereby the level of said protein is reduced upon addition or withdrawal of a factor from growth medium of said cell, wherein said reduction results in enhanced production of a metabolic product from said cell.

2. The cell of claim 1, wherein the enzymatic activity of said first moiety is catabolic enzymatic activity or anabolic enzymatic activity.

3. The cell of claim 1, wherein said first moiety is an enzyme selected from the group consisting of a kinase, an acetyl-CoA-producing enzyme, an enzyme that joins two carbon-containing reactants into a single carbon-containing product, an enzyme that acts downstream of glucose-6-phosphate in cellular metabolism, and an allosterically regulated enzyme.

4. The cell of claim 3, wherein said kinase is pyruvate kinase or shikimate kinase.

5. The cell of claim 3, wherein said acetyl-CoA-producing enzyme is pyruvate dehydrogenase.

6. The cell of claim 3, wherein said enzyme that joins two carbon-containing reactants into a single carbon-containing product is citrate synthase or DAHP synthase.

7. A cell of claim 1, wherein said second moiety differs from the sequence Ala-Ala-Asn-Asp-Glu-Asn-Tyr-Ala-Leu-Ala-Ala by at most four amino acid substitutions or deletions.

8. The cell of claim 7, wherein said second moiety comprises the sequence of any one of SEQ ID NOs: 1-2 and 4-10.

9. The cell of claim 1, wherein said regulatory system comprises a regulated promoter.

10. The cell of claim 9, wherein said promoter is selected from the group consisting of a lac operon promoter, a nitrogen-regulated promoter, a quorum sensing promoter, and a temperature-sensitive promoter.

11. The cell of claim 1, wherein said regulatory system controls synthesis of said protein.

12. The cell of claim 1, wherein said regulatory system controls synthesis of a factor that controls degradation of said protein.

13. The cell of claim 12, wherein said factor mediates recognition of said second moiety attached to said protein by cellular degradation enzymes.

14. The cell of claim 1, wherein said cell is a microbial cell.

15. The cell of claim 14, wherein said cell is a bacterial cell.

16. The cell of claim 14, wherein said cell is a fungal cell.

17. A method for producing a metabolic product, said method comprising:
(a) culturing in a suitable media the cell of claim 1 under conditions that allow production of said metabolic product, wherein a promoter of said regulatory system is repressed and wherein the production level of said metabolic product is greater than when said cell is cultured under conditions wherein said promoter is not repressed; and
(b) recovering said metabolic product from said cells or said media.

18. A method for producing a desired product from a microbe, comprising enhancing the inactivation of a protein in said microbe that contributes to the synthesis of one or more products that are not the desired product.


SEQUENCE LISTING (53 sequences)

SEQ ID NO: 1 - protein, 11 aa, Escherichia coli
AANDENYALAA

SEQ ID NO: 2 - protein, 11 aa, Artificial Sequence
  Description of Artificial Sequence Synthetic peptide
AANDENYADAS

SEQ ID NO: 3 - protein, 14 aa, Caulobacter crescentus
AANDNFAEEFAVAA

SEQ ID NO: 4 - protein, 11 aa, Escherichia coli
AANDENYADAA

SEQ ID NO: 5 - protein, 11 aa, Escherichia coli
AANDENYAAAA

SEQ ID NO: 6 - protein, 11 aa, Escherichia coli
AANDENYAVAA

SEQ ID NO: 7 - protein, 11 aa, Escherichia coli
AANDENYALDA

SEQ ID NO: 8 - protein, 11 aa, Escherichia coli
AANDENYALVA

SEQ ID NO: 9 - protein, 11 aa, Escherichia coli
AANDENYALAG

SEQ ID NO: 10 - protein, 11 aa, Escherichia coli
AANDENYALGG

SEQ ID NO: 11 - protein, 14 aa, Caulobacter crescentus
ADNDNFAEEFADAS

SEQ ID NO: 12 - protein, 14 aa, Artificial Sequence
  Description of Artificial Sequence Synthetic peptide
MTNTAKILNFGRAS

SEQ ID NO: 13 - protein, 11 aa, Escherichia coli
AANDENYALDD

SEQ ID NO: 14 - protein, 184 aa, Escherichia coli
MAEKRNIFLVGPMGAGKSTIGRQLAQQLNMEFYDSDQEIEKRTGADVGWVFDLEGEEGFRDREEKVINELTEKQGIVLATGGGSVKSRETRNRLSARGVVVYLETTIEKQLARTQRDKKRPLLHVETPPREVLEALANERNPLYEEIADVTIRTDDQSAKVVANQIIHMLESNAANDENYALAA

SEQ ID NO: 15 - protein, 189 aa, Artificial Sequence
  Description of Artificial Sequence Synthetic polypeptide
MAEKRNIFLVGPMGAGKSTIGRQLAQQLNMEFYDSDQEIEKRTGADVGWVFDLEGEEGFRDREEKVINELTEKQGIVLATGGGSVKSRETRNRLSARGVVVYLETTIEKQLARTQRDKKRPLLHVETPPREVLEALANERNPLYEEIADVTIRTDDQSAKVVANQIIHMLESNGGSGGAANDENYALAA

SEQ ID NO: 16 - protein, 187 aa, Escherichia coli
MTNTAKILNFGRASMAEKRNIFLVGPMGAGKSTIGRQLAQQLNMEFYDSDQEIEKRTGADVGWVFDLEGEEGFRDREEKVINELTEKQGIVLATGGGSVKSRETRNRLSARGVVVYLETTIEKQLARTQRDKKRPLLHVETPPREVLEALANERNPLYEEIADVTIRTDDQSAKVVANQIIHMLESN

SEQ ID NO: 17 - protein, 192 aa, Artificial Sequence
  Description of Artificial Sequence Synthetic polypeptide
MTNTAKILNFGRASGGSGGMAEKRNIFLVGPMGAGKSTIGRQLAQQLNMEFYDSDQEIEKRTGADVGWVFDLEGEEGFRDREEKVINELTEKQGIVLATGGGSVKSRETRNRLSARGVVVYLETTIEKQLARTQRDKKRPLLHVETPPREVLEALANERNPLYEEIADVTIRTDDQSAKVVANQIIHMLESN

SEQ ID NO: 18 - protein, 586 aa, Escherichia coli
MISGILASPGIAFGKALLLKEDEIVIDRKKISADQVDQEVERFLSGRAKASAQLETIKTKAGETFGEEKEAIFEGHIMLLEDEELEQEIIALIKDKHMTADAAAHEVIEGQASALEELDDEYLKERAADVRDIGKRLLRNILGLKIIDLSAIQDEVILVAADLTPSETAQLNLKKVLGFITDAGGRTSHTSIMARSLELPAIVGTGSVTSQVKNDDYLILDAVNNQVYVNPTNEVIDKMRAVQEQVASEKAELAKLKDLPAITLDGHQVEVCANIGTVRDVEGAERNGAEGVGLYRTEFLFMDRDALPTEEEQFAAYKAVAEACGSQAVIVRTMDIGGDKELPYMNFPKEENPFLGWRAIRIAMDRREILRDQLRAILRASAFGKLRIMFPMIISVEEVRALRKEIEIYKQELRDEGKAFDESIEIGVMVETPAAATIARHLAKEVDFFSIGTNDLTQYTLAVDRGNDMISHLYQPMSPSVLNLIKQVIDASHAEGKWTGMCGELAGDERATLLLLGMGLDEFSMSAISIPRIKKIIRNTNFEDAKVLAEQALAQPTTDELMTLVNKFIEEKTICAANDENYALAA

SEQ ID NO: 19 - protein, 481 aa, Escherichia coli
MKKTKIVCTIGPKTESEEMLAKMLDAGMNVMRLNFSHGDYAEHGQRIQNLRNVMSKTGKTAAILLDTKGPEIRTMKLEGGNDVSLKAGQTFTFTTDKSVIGNSEMVAVTYEGFTTDLSVGNTVLVDDGLIGMEVTAIEGNKVICKVLNNGDLGENKGVNLPGVSIALPALAEKDKQDLIFGCEQGVDFVAASFIRKRSDVIEIREHLKAHGGENIHIISKIENQEGLNNFDEILEASDGIMVARGDLGVEIPVEEVIFAQKMMIEKCIRARKVVITATQMLDSMIKNPRPTRAEAGDVANAILDGTDAVMLSGESAKGKYPLEAVSIMATICERTDRVMNSRLEFNNDNRKLRITEAVCRGAVETAEKLDAPLIVVATQGGKSARAVRKYFPDATILALTTNEKTAHQLVLSKGVVPQLVKEITSTDDFYRLGKELALQSGLAHKGDVVVMVSGALVPSGTTNTASVHVLAANDENYALAA

SEQ ID NO: 20 - protein, 438 aa, Escherichia coli
MADTKAKLTLNGDTAVELDVLKGTLGQDVIDIRTLGSKGVFTFDPGFTSTASCESKITFIDGDEGILLHRGFPIDQLATDSNYLEVCYILLNGEKPTQEQYDEFKTTVTRHTMIHEQITRLFHAFRRDSHPMAVMCGITGALAAFYHDSLDVNNPRHREIAAFRLLSKMPTMAAMCYKYSIGQPFVYPRNDLSYAGNFLNMMFSTPCEPYEVNPILERAMDRILILHADHEQNASTSTVRTAGSSGANPFACIAAGIASLWGPAHGGANEAALKMLEEISSVKHIPEFVRRAKDKNDSFRLMGFGHRVYKNYDPRATVMRETCHEVLKELGTKDDLLEVAMELENIALNDPYFIEKKLYPNVDFYSGIILKAMGIPSSMFTVIFAMARTVGWIAHWSEMHSDGMKIARPRQLYTGYEKRDFKSDIKRAANDENYALAA

SEQ ID NO: 21 - protein, 367 aa, Escherichia coli
MQKDALNNVHITDEQVLMTPEQLKAAFPLSLQQEAQIADSRKSISDIIAGRDPRLLVVCGPCSIHDPETALEYARRFKALAAEVSDSLYLVMRVYFEKPRTTVGWKGLINDPHMDGSFDVEAGLQIARKLLLELVNMGLPLATEALDPNSPQYLGDLFSWSAIGARTTESQTHREMASGLSMPVGFKNGTDGSLATAINAMRAAAQPHRFVGINQAGQVALLQTQGNPDGHVILRGGKAPNYSPADVAQCEKEMEQAGLRPSLMVDCSHGNSNKDYRRQPAVAESVVAQIKDGNRSIIGLMIESNIHEGNQSSEQPRSEMKYGVSVTDACISWEMTDALLREIHQDLNGQLTARVAAANDENYALAA

SEQ ID NO: 22 - protein, 424 aa, Escherichia coli
MTDKRKDGSGKLLYCSFCGKSQHEVRKLIAGPSVYICDECVDLCNDIIREEIKEVAPHRERSALPTPHEIRNHLDDYVIGQEQAKKVLAVAVYNHYKRLRNGDTSNGVELGKSNILLIGPTGSGKTLLAETLARLLDVPFTMADATTLTEAGYVGEDVENIIQKLLQKCDYDVQKAQRGIVYIDEIDKISRKSDNPSITRDVSGEGVQQALLKLIEGTVAAVPPQGGRKHPQQEFLQVDTSKILFICGGAFAGLDKVISHRVETGSGIGFGATVKAKSDKASEGELLAQVEPEDLIKFGLIPEFIGRLPVVATLNELSEEALIQILKEPKNALTKQYQALFNLEGVDLEFRDEALDAIAKKAMARKTGARGLRSIVEAALLDTMYDLPSMEDVEKVVIDESVIDGQSKPLLIYGKPEAQQASGE

SEQ ID NO: 23 - protein, 758 aa, Escherichia coli
MLNQELELSLNMAFARAREHRHEFMTVEHLLLALLSNPSAREALEACSVDLVALRQELEAFIEQTTPVLPASEEERDTQPTLSFQRVLQRAVFHVQSSGRNEVTGANVLVAIFSEQESQAAYLLRKHEVSRLDVVNFISHGTRKDEPTQSSDPGSQPNSEEQAGGEERMENFTTNLNQLARVGGIDPLIGREKELERAIQVLCRRRKNNPLLVGESGVGKTAIAEGLAWRIVQGDVPEVMADCTIYSLDIGSLLAGTKYRGDFEKRFKALLKQLEQDTNSILFIDEIHTIIGAGAASGGQVDAANLIKPLLSSGKIRVIGSTTYQEFSNIFEKDRALARRFQKIDITEPSIEETVQIINGLKPKYEAHHDVRYTAKAVRAAVELAVKYINDRHLPDKAIDVIDEAGARARLMPVSKRKKTVNVADIESVVARIARIPEKSVSQSDRDTLKNLGDRLKMLVFGQDKAIEALTEAIKMARAGLGHEHKPVGSFLFAGPTGVGKTEVTVQLSKALGIELLRFDMSEYMERHTVSRLIGAPPGYVGFDQGGLLTDAVIKHPHAVLLLDEIEKAHPDVFNILLQVMDNGTLTDNNGRKADFRNVVLVMTTNAGVRETERKSIGLIHQDNSTDAMEEIKKIFTPEFRNRLDNIIWFDHLSTDVIHQVVDKFIVELQVQLDQKGVSLEVSQEARNWLAEKGYDRAMGARPMARVIQDNLKKPLANELLFGSLVDGGQVTVALDKEKNELTYGFQSAQKHKAEAAH

SEQ ID NO: 24 - protein, 207 aa, Escherichia coli
MSYSGERDNFAPHMALVPMVIEQTSRGERSFDIYSRLLKERVIFLTGQVEDHMANLIVAQMLFLEAENPEKDIYLYINSPGGVITAGMSIYDTMQFIKPDVSTICMGQAASMGAFLLTAGAKGKRFCLPNSRVMIHQPLGGYQGQATDIEIHAREILKVKGRMNELMALHTGQSLEQIERDTERDRFLSAPEAVEYGLVDSILTHRN

SEQ ID NO: 25 - protein, 165 aa, Escherichia coli [sequence omitted]

SEQ ID NO: 26 - protein, 106 aa, Escherichia coli
MGKTNDWLDFDQLAEEKVRDALKPPSMYKVILVNDDYTPMEFVIDVLQKFFSYDVERATQLMLAVHYQGKAICGVFTAEVAETKVAMVNKYARENEHPLLCTLEKA

SEQ ID NO: 27 - protein, 162 aa, Caulobacter crescentus
MSQTEPPEDLMQYEAMAQDALRGVVKAALKKAAAPGGLPEPHHLYITFKTKAAGVSGPQDLLSKYPDEMTIVLQHQYWDLAPGETFFSVTLKFGGQPKRLSVPYAALTRFYDPSVQFALQFSAPEIIEDEPEPDPEPEDKANQGASGDEGPKIVSLDQFRKK

SEQ ID NO: 28 - DNA, 1252 bp, Artificial Sequence
  Description of Artificial Sequence Synthetic polynucleotide
gaagttctggaagcgttggccaatgaacgcaatccgctgtatgaagagattgccgacgtgaccattcgtactgatgatcaaagcgctaaagtggttgcaaaccagattattcacatgctggaaagcaacgcagctaacgatgaaaactacagcgaaaactatgctgacgctagctaatactagagctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcccgtccgcgcttaaactccaacatggacgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtccgtctcaactggctgacggagtttatgcctctcccgaccatcaagcattttatccgtactcctgatgatgcgtggttactcaccaccgcgattcctgggaaaacagccttccaggtattagaagaatatcctgattcaggtgaaaatattgttgatgcgctggccgtgttcctgcgccggttacattcgattcctgtttgtaattgtccttttaacagcgatcgtgtatttcgtcttgctcaggcgcaatcacgcatgaataacggtttggttgatgcgagtgattttgatgacgagcgtaatggctggcctgttgaacaagtctggaaagaaatgcacaagctcttgccattctcaccggattcagtcgtcactcatggtgatttctcacttgataaccttatttttgacgaggggaaattaataggttgtattgatgttggacgggtcggaatcgcagaccgttaccaggaccttgccattctttggaactgcctcggtgagttttctccttcattacagaaacggctttttcaaaaatatggtattgataatcctgatatgaataaattgcagtttcatttgatgctcgatgagtttttctaataattctggctttatatacactcgtctgcgggtacagtaattaaggtggatgtcgcgttatggagaggattgtcgttactctcggggaacgtagttacccaat

SEQ ID NO: 29 - DNA, 58 bp, Artificial Sequence
  Description of Artificial Sequence Synthetic oligonucleotide
ggccgcttctagagtcacacaggaaagtactagatggcagagaaacgcaatatctttc

SEQ ID NO: 30 - DNA, 86 bp, Artificial Sequence
  Description of Artificial Sequence Synthetic oligonucleotide
ggccctgcagcggccgctactagtattaagcagccagagcataattttcatcgttagctgcgttgctttccagcatgtgaataatc

SEQ ID NO: 31 - DNA, 2738 bp, Artificial Sequence
  Description of Artificial Sequence Synthetic polynucleotide
tactagtagcggccgctgcaggagtcactaagggttagttagttagattagcagaaagtcaaaagcctccgaccggaggcttttgactaaaacttcccttggggttatcattggggctcactcaaaggcggtaatcagataaaaaaaatccttagctttcgctaaggatgatttctgctagagatggaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgtttactagaggttgatcgggcacgtaagaggttccaactttcaccataatgaaataagatcactaccgggcgtattttttgagttatcgagattttcaggagctaaggaagctaaaatggagaaaaaaatcacgggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaacgctcacccggagtttcgtatggccatgaaagacggtgagctggtgatctgggatagtgttcacccttgttacaccgttttccatgagcaaactgaaacgttttcgtccctctggagtgaataccacgacgatttccggcagtttctccacatatattcgcaagatgtggcgtgttacggtgaaaacctggcctatttccctaaagggtttattgagaatatgttttttgtctcagccaatccctgggtgagtttcaccagttttgatttaaacgtggccaatatggacaacttcttcgcccccgttttcacgatgggcaaatattatacgcaaggcgacaaggtgctgatgccgctggcgatccaggttcatcatgccgtttgtgatggcttccatgtcggccgcatgcttaatgaattacaacagtactgtgatgagtggcagggcggggcgtaataatactagctccggcaaaaaaacgggcaaggtgtcaccaccctgccctttttctttaaaaccgaaaagattacttcgcgtttgccacctgacgtctaagaaaaggaatattcagcaatttgcccgtgccgaagaaaggcccacccgtgaaggtgagccagtgagttgattgctacgtaattagttagttagcccttagtgactcgaattcgcggccgcttctagag

SEQ ID NO: 32 - DNA, 1061 bp, Artificial Sequence
  Description of Artificial Sequence Synthetic polynucleotide
tccctatcagtgatagagattgacatccctatcagtgatagagatactgagcactactagagaaagaggagaaatactagatgaaaaacataaatgccgacgacacatacagaataattaataaaattaaagcttgtagaagcaataatgatattaatcaatgcttatctgatatgactaaaatggtacattgtgaatattatttactcgcgatcatttatcctcattctatggttaaatctgatatttcaatcctagataattaccctaaaaaatggaggcaatattatgatgacgctaatttaataaaatatgatcctatagtagattattctaactccaatcattcaccaattaattggaatatatttgaaaacaatgctgtaaataaaaaatctccaaatgtaattaaagaagcgaaaacatcaggtcttatcactgggtttagtttccctattcatacggctaacaatggcttcggaatgcttagttttgcacattcagaaaaagacaactatatagatagtttatttttacatgcgtgtatgaacataccattaattgttccttctctagttgataattatcgaaaaataaatatagcaaataataaatcaaacaacgatttaaccaaaagagaaaaagaatgtttagcgtgggcatgcgaaggaaaaagctcttgggatatttcaaaaatattaggttgcagtgagcgtactgtcactttccatttaaccaatgcgcaaatgaaactcaatacaacaaaccgctgccaaagtatttctaaagcaattttaacaggagcaattgattgcccatactttaaaaattaataacactgatagtgctagtgtagatcactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacctgtaggatcgtacaggtttacgcaagaaaatggtttgttatagtcgaataaa

SEQ ID NO: 33 - DNA, 200 bp, Artificial Sequence
  Description of Artificial Sequence Synthetic polynucleotide
caatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacaca

SEQ ID NO: 34 - DNA, 13 bp, Artificial Sequence
  Description of Artificial Sequence Synthetic oligonucleotide
tcacacaggaaag

SEQ ID NO: 35 - DNA, 519 bp, Artificial Sequence
  Description of Artificial Sequence Synthetic polynucleotide
atggcagagaaacgcaatatctttctggttgggcctatgggtgccggaaaaagcactattgggcgccagttagctcaacaactcaatatggaattttacgattccgatcaagagattgagaaacgaaccggagctgatgtgggctgggttttcgatttagaaggcgaagaaggcttccgcgatcgcgaagaaaaggtcatcaatgagttgaccgagaaacagggtattgtgctggctactggcggcggctctgtgaaatcccgtgaaacgcgtaaccgtctttccgctcgtggcgttgtcgtttatcttgaaacgaccatcgaaaagcaacttgcacgcacgcagcgtgataaaaaacgcccgttgctgcacgttgaaacaccgccgcgtgaagttctggaagcgttggccaatgaacgcaatccgctgtatgaagagattgccgacgtgaccattcgtactgatgatcaaagcgctaaagtggttgcaaaccagattattcacatgctggaaagcaac

SEQ ID NO: 36 - DNA, 498 bp, Artificial Sequence
  Description of Artificial Sequence Synthetic polynucleotide
atggatttgtcacagctaacaccacgtcgtccctatctgctgcgtgcattctatgagtggttgctggataaccagctcacgccgcacctggtggtggatgtgacgctccctggcgtgcaggttcctatggaatatgcgcgtgacgggcaaatcgtactcaacattgcgccgcgtgctgtcggcaatctggaactggcgaatgatgaggtgcgctttaacgcgcgctttggtggcattccgcgtcaggtttctgtgccgctggctgccgtgctggctatctacgcccgtgaaaatggcgcaggcacgatgtttgagcctgaagctgcctacgatgaagataccagcatcatgaatgatgaagaggcatcggcagacaacgaaaccgttatgtcggttattgatggcgacaagccagatcacgatgatgacactcatcctgacgatgaacctccgcagccaccacgcggtggtcgaccggcattacgcgttgtgaagtaa

SEQ ID NO: 37 - DNA, 36 bp, Artificial Sequence
  Description of Artificial Sequence Synthetic oligonucleotide
gcagctaacgatgaaaattatgctctggctgcttaa

SEQ ID NO: 38 - DNA, 36 bp, Artificial Sequence
  Description of Artificial Sequence Synthetic oligonucleotide
gcagctaacgatgaaaattatgctctggttgcttaa

SEQ ID NO: 39 - DNA, 36 bp, Artificial Sequence
  Description of Artificial Sequence Synthetic oligonucleotide
gcagctaacgatgaaaattatgctgacgctagctaa

SEQ ID NO: 40 - DNA, 36 bp, Artificial Sequence
  Description of Artificial Sequence Synthetic oligonucleotide
gcagctaacgatgaaaattatgctctggacgactaa

SEQ ID NO: 41 - DNA, 4379 bp, Artificial Sequence
  Description of Artificial Sequence Synthetic polynucleotide
gaattcgcggccgcttctagtccctatcagtgatagagattgacatccctatcagtgatagagatactgagcactactagagaaagaggagaaatactagatgaaaaacataaatgccgacgacacatacagaataattaataaaattaaagcttgtagaagcaataatgatattaatcaatgcttatctgatatgactaaaatggtacattgtgaatattatttactcgcgatcatttatcctcattctatggttaaatctgatatttcaatcctagataattaccctaaaaaatggaggcaatattatgatgacgctaatttaataaaatatgatcctatagtagattattctaactccaatcattcaccaattaattggaatatatttgaaaacaatgctgtaaataaaaaatctccaaatgtaattaaagaagcgaaaacatcaggtcttatcactgggtttagtttccctattcatacggctaacaatggcttcggaatgcttagttttgcacattcagaaaaagacaactatatagatagtttatttttacatgcgtgtatgaacataccattaattgttccttctctagttgataattatcgaaaaataaatatagcaaataataaatcaaacaacgatttaaccaaaagagaaaaagaatgtttagcgtgggcatgcgaaggaaaaagctcttgggatatttcaaaaatattaggttgcagtgagcgtactgtcactttccatttaaccaatgcgcaaatgaaactcaatacaacaaaccgctgccaaagtatttctaaagcaattttaacaggagcaattgattgcccatactttaaaaattaataacactgatagtgctagtgtagatcactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacctgtaggatcgtacaggtttacgcaagaaaatggtttgttatagtcgaataaatactagagtcacacaggaaagtactagatggcagagaaacgcaatatctttctggttgggcctatgggtgccggaaaaagcactattgggcgccagttagctcaacaactcaatatggaattttacgattccgatcaagagattgagaaacgaaccggagctgatgtgggctgggttttcgatttagaaggcgaagaaggcttccgcgatcgcgaagaaaaggtcatcaatgagttgaccgagaaacagggtattgtgctggctactggcggcggctctgtgaaatcccgtgaaacgcgtaaccgtctttccgctcgtggcgttgtcgtttatcttgaaacgaccatcgaaaagcaacttgcacgcacgcagcgtgataaaaaacgcccgttgctgcacgttgaaacaccgccgcgtgaagttctggaagcgttggccaatgaacgcaatccgctgtatgaagagattgccgacgtgaccattcgtactgatgatcaaagcgctaaagtggttgcaaaccagattattcacatgctggaaagcaacgcagctaacgatgaaaattatgctctggttgcttaatactagtagcggccgctgcaggagtcactaagggttagttagttagattagcagaaagtcaaaagcctccgaccggaggcttttgactaaaacttcccttggggttatcattggggctcactcaaaggcggtaatcagataaaaaaaatccttagctttcgctaaggatgatttctgctagagatggaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgtttactagaggttgatcgggcacgtaagaggttccaactttcaccataatgaaataagatcactaccgggcgtattttttgagttatcgagattttcaggagctaaggaagctaaaatggagaaaaaaatcacgggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaacgctcacccggagtttcgtatggccatgaaagacggtgagctggtgatctgggatagtgttcacccttgttacaccgttttccatgagcaaactgaaacgttttcgtccctctggagtgaataccacgacgatttccggcagtttctccacatatattcgcaagatgtggcgtgttacggtgaaaacctggcctatttccctaaagggtttattgagaatatgttttttgtctcagccaatccctgggtgagtttcaccagttttgatttaaacgtggccaatatggacaacttcttcgcccccgttttcacgatgggcaaatattatacgcaaggcgacaaggtgctgatgccgctggcgatccaggttcatcatgccgtttgtgatggcttccatgtcggccgcatgcttaatgaattacaacagtactgtgatgagtggcagggcggggcgtaataatactagctccggcaaaaaaacgggcaaggtgtcaccaccctgccctttttctttaaaaccgaaaagattacttcgcgtttgccacctgacgtctaagaaaaggaatattcagcaatttgcccgtgccgaagaaaggcccacccgtgaaggtgagccagtgagttgattgctacgtaattagttagttagcccttagtgactc

SEQ ID NO: 42 - DNA, 1137 bp, Corynebacterium glutamicum
atgacgtcattagagttcacagtaacccgtaccgaaaatccgacgtcacccgatcgtctgaaggaaattcttgccgcaccgaagttcggtaagttcttcaccgaccacatggtgaccattgactggaacgagtcggaaggctggcacaacgcccaattagtgccatacgcgccgattcctatggatcctgccaccaccgtattccactacggacaggcaatttttgagggaattaaggcctaccgccattcggacgaaaccatcaagactttccgtcctgatgaaaacgccgagcgtatgcagcgttcagcagctcgaatggcaatgccacagttgccaaccgaggactttattaaagcacttgaactgctggtagacgcggatcaggattgggttcctgagtacggcggagaagcttccctctacctgcgcccattcatgatctccaccgaaattggcttgggtgtcagcccagctgatgcctacaagttcctggtcatcgcatccccagtcggcgcttacttcaccggtggaatcaagcctgtttccgtctggctgagcgaagattacgtccgcgctgcacccggcggaactggtgacgccaaatttgctggcaactacgcggcttctttgcttgcccagtcccaggctgcggaaaagggctgtgaccaggtcgtatggttggatgccatcgagcacaagtacatcgaagaaatgggtggcatgaaccttgggttcatctaccgcaacggcgaccaagtcaagctagtcacccctgaactttccggctcactacttccaggcatcacccgcaagtcacttctacaagtagcacgcgacttgggatacgaagtagaagagcgaaagatcaccaccaccgagtgggaagaagacgcaaagtctggcgccatgaccgaggcatttgcttgcggtactgcagctgttatcacccctgttggcaccgtgaaatcagctcacggcaccttcgaagtgaacaacaatgaagtcggagaaatcacgatgaagcttcgtgaaaccctcaccggaattcagcaaggaaacgttgaagaccaaaacggatggctttacccactggttggcgcagctaacgatgaaaattatgctctggtggcttaa

SEQ ID NO: 43 - DNA, 843 bp, Corynebacterium glutamicum
atgtcaggcattgatgcaaagaaaatccgcacccgtcatttccgcgaagctaaagtaaacggccagaaagtttcggttctcaccagctatgatgcgctttcggcgcgcatttttgatgaggctggcgtcgatatgctccttgttggtgattccgctgccaacgttgtgctgggtcgcgataccaccttgtcgatcaccttggatgagatgattgtgctggccaaggcggtgacgatcgctacgaagcgtgcgcttgtggtggttgatctgccgtttggtacctatgaggtgagcccaaatcaggcggtggagtccgcgatccgggtcatgcgtgaaacgggtgcggctgcggtgaagatcgagggtggcgtggagatcgcgcagacgattcgacgcattgttgatgctggaattccggttgtcggccacatcgggtacaccccgcagtccgagcattccttgggcggccacgtggttcagggtcgtggcgcgagttctggaaagctcatcgccgatgcccgcgcgttggagcaggcgggtgcgtttgcggttgtgttggagatggttccagcagaggcagcgcgcgaggttaccgaggatctttccatcaccactatcggaatcggtgccggcaatggcacagatgggcaggttttggtgtggcaggatgccttcggcctcaaccgcggcaagaagccacgcttcgtccgcgagtacgccaccttgggcgattccttgcacgacgccgcgcaggcctacatcgccgatatccacgcgggtaccttcccaggcgaagcggagtcctttgcagctaacgatgaaaattatgctctgggcggctaa

SEQ ID NO: 44 - DNA, 1950 bp, Corynebacterium glutamicum
atgcttcaccacatgacttcgcgtgcgaatctacttcttcttcgccgcggcgggtcccagaggtctatgtctcctaacgatgcattcatctccgcacctgccaagatcgaaaccccagttgggcctcgcaacgaaggccagccagcatggaataagcagcgtggctcctcaatgccagttaaccgctacatgcctttcgaggttgaggtagaagatatttctctgccggaccgcacttggccagataaaaaaatcaccgttgcacctcagtggtgtgctgttgacctgcgtgacggcaaccaggctctgattgatccgatgtctcctgagcgtaagcgccgcatgtttgagctgctggttcagatgggcttcaaagaaatcgaggtcggtttcccttcagcttcccagactgattttgatttcgttcgtgagatcatcgaaaagggcatgatccctgacgatgtcaccattcaggttctggttcaggctcgtgagcacctgattcgccgtacttttgaagcttgcgaaggcgcaaaaaacgttatcgtgcacttctacaactccacctccatcctgcagcgcaacgtggtgttccgcatggacaaggtgcaggtgaagaagctggctaccgatgccgctgaactaatcaagaccatcgctcaggattacccagacaccaactggcgctggcagtactcccctgagtccttcaccggcactgaggttgagtacgccaaggaagttgtggacgcagttgttgaggtcatggatccaactcctgagaacccaatgatcatcaacctgccttccaccgttgagatgatcacccctaacgtttacgcagactccattgaatggatgcaccgcaatctaaaccgtcgtgattccattatcctgtccctgcacccgcacaatgaccgtggcaccggcgttggcgcagctgagctgggctacatggctggcgctgaccgcatcgaaggctgcctgttcggcaacggcgagcgcaccggcaacgtctgcctggtcaccctggcactgaacatgctgacccagggcgttgaccctcagctggacttcaccgatatacgccagatccgcagcaccgttgaatactgcaaccagctgcgcgttcctgagcgccacccatacggcggtgacctggtcttcaccgctttctccggttcccaccaggacgctgtgaacaagggtctggacgccatggctgccaaggttcagccaggtgctagctccactgaagtttcttgggagcagctgcgcgacaccgaatgggaggttccttacctgcctatcgatccaaaggatgtcggtcgcgactacgaggctgttatccgcgtgaactcccagtccggcaagggcggcgttgcttacatcatgaagaccgatcacggtctgcagatccctcgctccatgcaggttgagttctccaccgttgtccagaacgtcaccgacgctgagggcggcgaggtcaactccaaggcaatgtgggatatcttcgccaccgagtacctggagcgcaccgcaccagttgagcagatcgcgctgcgcgtcgagaacgctcagaccgaaaacgaggatgcatccatcaccgccgagctcatccacaacggcaaggacgtcaccgtcgatggccgcggcaacggcccactggccgcttacgccaacgcgctggagaagctgggcatcgacgttgagatccaggaatacaaccagcacgcccgcacctcgggcgacgatgcagaagcagccgcctacgtgctggctgaggtcaacggccgcaaggtctggggcgtcggcatcgctggctccatcacctacgcttcgctgaaggcagtgacctccgccgtaaaccgcgcgctggacgtcaaccacgaggcagtcctggctggcggcgttgcagctaacgatgaaaattatgctctggctggctaa

SEQ ID NO: 45 - protein, 378 aa, Corynebacterium glutamicum
MTSLEFTVTRTENPTSPDRLKEILAAPKFGKFFTDHMVTIDWNESEGWHNAQLVPYAPIPMDPATTVFHYGQAIFEGIKAYRHSDETIKTFRPDENAERMQRSAARMAMPQLPTEDFIKALELLVDADQDWVPEYGGEASLYLRPFMISTEIGLGVSPADAYKFLVIASPVGAYFTGGIKPVSVWLSEDYVRAAPGGTGDAKFAGNYAASLLAQSQAAEKGCDQVVWLDAIEHKYIEEMGGMNLGFIYRNGDQVKLVTPELSGSLLPGITRKSLLQVARDLGYEVEERKITTTEWEEDAKSGAMTEAFACGTAAVITPVGTVKSAHGTFEVNNNEVGEITMKLRETLTGIQQGNVEDQNGWLYPLVGAANDENYALVA

SEQ ID NO: 46 - protein, 280 aa, Corynebacterium glutamicum
MSGIDAKKIRTRHFREAKVNGQKVSVLTSYDALSARIFDEAGVDMLLVGDSAANVVLGRDTTLSITLDEMIVLAKAVTIATKRALVVVDLPFGTYEVSPNQAVESAIRVMRETGAAAVKIEGGVEIAQTIRRIVDAGIPVVGHIGYTPQSEHSLGGHVVQGRGASSGKLIADARALEQAGAFAVVLEMVPAEAAREVTEDLSITTIGIGAGNGTDGQVLVWQDAFGLNRGKKPRFVREYATLGDSLHDAAQAYIADIHAGTFPGEAESFAANDENYALGG

SEQ ID NO: 47 - protein, 649 aa, Corynebacterium glutamicum
MLHHMTSRANLLLLRRGGSQRSMSPNDAFISAPAKIETPVGPRNEGQPAWNKQRGSSMPVNRYMPFEVEVEDISLPDRTWPDKKITVAPQWCAVDLRDGNQALIDPMSPERKRRMFELLVQMGFKEIEVGFPSASQTDFDFVREIIEKGMIPDDVTIQVLVQAREHLIRRTFEACEGAKNVIVHFYNSTSILQRNVVFRMDKVQVKKLATDAAELIKTIAQDYPDTNWRWQYSPESFTGTEVEYAKEVVDAVVEVMDPTPENPMIINLPSTVEMITPNVYADSIEWMHRNLNRRDSIILSLHPHNDRGTGVGAAELGYMAGADRIEGCLFGNGERTGNVCLVTLALNMLTQGVDPQLDFTDIRQIRSTVEYCNQLRVPERHPYGGDLVFTAFSGSHQDAVNKGLDAMAAKVQPGASSTEVSWEQLRDTEWEVPYLPIDPKDVGRDYEAVIRVNSQSGKGGVAYIMKTDHGLQIPRSMQVEFSTVVQNVTDAEGGEVNSKAMWDIFATEYLERTAPVEQIALRVENAQTENEDASITAELIHNGKDVTVDGRGNGPLAAYANALEKLGIDVEIQEYNQHARTSGDDAEAAAYVLAEVNGRKVWGVGIAGSITYASLKAVTSAVNRALDVNHEAVLAGGVAANDENYALAG

SEQ ID NO: 48 - DNA, 963 bp, Escherichia coli
atgaccacgaagaaagctgattacatttggttcaatggggagatggttcgctgggaagacgcgaaggtgcatgtgatgtcgcacgcgctgcactatggcacttcggtttttgaaggcatccgttgctacgactcgcacaaaggaccggttgtattccgccatcgtgagcatatgcagcgtctgcatgactccgccaaaatctatcgcttcccggtttcgcagagcattgatgagctgatggaagcttgtcgtgacgtgatccgcaaaaacaatctcaccagcgcctatatccgtccgctgatcttcgtcggtgatgttggcatgggagtaaacccgccagcgggatactcaaccgacgtgattatcgctgctttcccgtggggagcgtatctgggcgcagaagcgctggagcaggggatcgatgcgatggtttcctcctggaaccgcgcagcaccaaacaccatcccgacggcggcaaaagccggtggtaactacctctcttccctgctggtgggtagcgaagcgcgccgccacggttatcaggaaggtatcgcgctggatgtgaacggttatatctctgaaggcgcaggcgaaaacctgtttgaagtgaaagatggtgtgctgttcaccccaccgttcacctcctccgcgctgccgggtattacccgtgatgccatcatcaaactggcgaaagagctgggaattgaagtacgtgagcaggtgctgtcgcgcgaatccctgtacctggcggatgaagtgtttatgtccggtacggcggcagaaatcacgccagtgcgcagcgtagacggtattcaggttggcgaaggccgttgtggcccggttaccaaacgcattcagcaagccttcttcggcctcttcactggcgaaaccgaagataaatggggctggttagatcaagttaatcaagcagctaacgatgaaaattatgctctggtggcttaa

SEQ ID NO: 49 - DNA, 828 bp, Escherichia coli
atgaaaccgaccaccatctccttactgcagaagtacaaacaggaaaaaaaacgtttcgcgaccatcaccgcttatgactatagcttcgccaaactctttgctgatgaagggcttaacgtcatgctggtgggcgattcgctgggcatgacggttcaggggcacgactccaccctgccagttaccgttgccgatatcgcctaccacactgccgccgtacgtcgcggcgcaccaaactgcctgctgctggctgacctgccgtttatggcgtatgccacgccggaacaagccttcgaaaacgccgcaacggttatgcgtgccggtgctaacatggtcaaaattgaaggcggtgagtggctggtagaaaccgtacaaatgctgaccgaacgtgccgttcctgtatgtggtcacttaggtttaacaccacagtcagtgaatattttcggtggctacaaagttcaggggcgcggcgatgaagcgggcgatcaactgctcagcgatgcattagccttagaagctgctggggcacagctgctggtgctggaatgcgtgccggttgaactggcaaaacgtattaccgaagcactggcgatcccggttattggcattggcgcaggcaacgtcactgacgggcagatcctcgtgatgcacgacgcctttggtattaccggcggtcacattcctaaattcgctaaaaatttcctcgccgaaacgggcgacatccgcgcggctgtgcggcagtatatggctgaagtggagtccggcgtttatccgggcgaagaacacagtttccatgcagctaacgatgaaaattatgctctgggcggctaa

SEQ ID NO: 50 - DNA, 1605 bp, Escherichia coli
atgagccagcaagtcattattttcgataccacattgcgcgacggtgaacaggcgttacaggcaagcttgagtgtgaaagaaaaactgcaaattgcgctggcccttgagcgtatgggtgttgacgtgatggaagtcggtttccccgtctcttcgccgggcgattttgaatcggtgcaaaccatcgcccgccaggttaaaaacagccgcgtatgtgcgttagctcgctgcgtggaaaaagatatcgacgtggcggccgaatccctgaaagtcgccgaagccttccgtattcatacctttattgccacttcgccaatgcacatcgccaccaagctgcgcagcacgctggacgaggtgatcgaacgcgctatctatatggtgaaacgcgcccgtaattacaccgatgatgttgaattttcttgcgaagatgccgggcgtacacccattgccgatctggcgcgagtggtcgaagcggcgattaatgccggtgccaccaccatcaacattccggacaccgtgggctacaccatgccgtttgagttcgccggaatcatcagcggcctgtatgaacgcgtgcctaacatcgacaaagccattatctccgtacatacccacgacgatttgggcctggcggtcggaaactcactggcggcggtacatgccggtgcacgccaggtggaaggcgcaatgaacgggatcggcgagcgtgccggaaactgttccctggaagaagtcatcatggcgatcaaagttcgtaaggatattctcaacgtccacaccgccattaatcaccaggagatatggcgcaccagccagttagttagccagatttgtaatatgccgatcccggcaaacaaagccattgttggcagcggcgcattcgcacactcctccggtatacaccaggatggcgtgctgaaaaaccgcgaaaactacgaaatcatgacaccagaatctattggtctgaaccaaatccagctgaatctgacctctcgttcggggcgtgcggcggtgaaacatcgcatggatgagatggggtataaagaaagtgaatataatttagacaatttgtacgatgctttcctgaagctggcggacaaaaaaggtcaggtgtttgattacgatctggaggcgctggccttcatcggtaagcagcaagaagagccggagcatttccgtctggattacttcagcgtgcagtctggctctaacgatatcgccaccgccgccgtcaaactggcctgtggcgaagaagtcaaagcagaagccgccaacggtaacggtccggtcgatgccgtctatcaggcaattaaccgcatcactgaatataacgtcgaactggtgaaatacagcctgaccgccaaaggccacggtaaagatgcgctgggtcaggtggatatcgtcgctaactacaacggtcgccgcttccacggcgtcggcctggctaccgatattgtcgagtcatctgccaaagccatggtgcacgttctgaacaatatctggcgtgccgcagaagtcgaaaaagagttgcaacgcaaagctcaacacaacgaaaacaacaaggaaaccgtggcagctaacgatgaaaattatgctctggctggctga

SEQ ID NO: 51 - protein, 320 aa, Escherichia coli
MTTKKADYIWFNGEMVRWEDAKVHVMSHALHYGTSVFEGIRCYDSHKGPVVFRHREHMQRLHDSAKIYRFPVSQSIDELMEACRDVIRKNNLTSAYIRPLIFVGDVGMGVNPPAGYSTDVIIAAFPWGAYLGAEALEQGIDAMVSSWNRAAPNTIPTAAKAGGNYLSSLLVGSEARRHGYQEGIALDVNGYISEGAGENLFEVKDGVLFTPPFTSSALPGITRDAIIKLAKELGIEVREQVLSRESLYLADEVFMSGTAAEITPVRSVDGIQVGEGRCGPVTKRIQQAFFGLFTGETEDKWGWLDQVNQAANDENYALVA

SEQ ID NO: 52 - protein, 275 aa, Escherichia coli
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGGEWLVETVQMLTERAVPVCGHLGLTPQSVNIFGGYKVQGRGDEAGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFHAANDENYALGG

SEQ ID NO: 53 - protein, 534 aa, Escherichia coli
MSQQVIIFDTTLRDGEQALQASLSVKEKLQIALALERMGVDVMEVGFPVSSPGDFESVQTIARQVKNSRVCALARCVEKDIDVAAESLKVAEAFRIHTFIATSPMHIATKLRSTLDEVIERAIYMVKRARNYTDDVEFSCEDAGRTPIADLARVVEAAINAGATTINIPDTVGYTMPFEFAGIISGLYERVPNIDKAIISVHTHDDLGLAVGNSLAAVHAGARQVEGAMNGIGERAGNCSLEEVIMAIKVRKDILNVHTAINHQEIWRTSQLVSQICNMPIPANKAIVGSGAFAHSSGIHQDGVLKNRENYEIMTPESIGLNQIQLNLTSRSGRAAVKHRMDEMGYKESEYNLDNLYDAFLKLADKKGQVFDYDLEALAFIGKQQEEPEHFRLDYFSVQSGSNDIATAAVKLACGEEVKAEAANGNGPVDAVYQAINRITEYNVELVKYSLTAKGHGKDALGQVDIVANYNGRRFHGVGLATDIVESSAKAMVHVLNNIWRAAEVEKELQRKAQHNENNKETVAANDENYALAG